(19) United States
(12) Patent Application Publication
(10) Pub. No.: US 2023/0265205 A1
(43) Pub. Date: Aug. 24, 2023

(54) METABOLISM-BASED CHIMERIC ANTIGEN RECEPTORS AND METHODS OF TREATMENT

(71) Applicant: Arizona Board of Regents on behalf of Arizona State University, Scottsdale, AZ (US)

(72) Inventors: Abhinav ACHARYA, Scottsdale, AZ (US); Mamta WANKHEDE, Scottsdale, AZ (US)

(21) Appl. No.: 18/041,123

(22) PCT Filed: Aug. 9, 2021
(86) PCT No.: PCT/US21/45183

§ 371 Date: Feb. 9, 2023

Related U.S. Application Data
(60) Provisional application No. 63/147,448, filed on Feb. 9, 2021.
(60) Provisional application No. 63/063,684, filed on Aug. 10, 2020.

(51) Int. Cl. C07K 16/28; C07K 16/22; C07K 16/30; A61K 31/70; A61K 31/194; C12N 15/63; A61P 35/00

(52) U.S. Cl. CPC C07K 16/2896; C07K 16/22; C07K 16/3092; A61K 31/70; A61K 31/194; C12N 15/63; A61P 35/00

(57) ABSTRACT

Disclosed are compositions comprising chimeric antigen receptors (CARs) and related methods of use in cancer immunotherapy. Compositions include reprogrammed immune cells (e.g., macrophages, neutrophils, dendritic cells, and T cells) that are metabolically fit for tumor microenvironments. The engineered immune cells are reprogrammed to express one or more of recombinant CARs at their cell surfaces and are loaded with glycolysis accelerating metabolites (e.g., F16BP or succinate). Methods of treating a subject with a condition, such as cancer, are also disclosed and include administering an effective amount of a composition comprising an engineered immune cell to a subject in need thereof.

### CROSS REFERENCE TO RELATED APPLICATIONS

This application claims priority to and the benefit of U.S. Provisional Patent Application No. 63/063,684 filed Aug. 10, 2020, and U.S. Provisional Patent Application No. 63/147,448 filed Feb. 9, 2021, both of which are incorporated herein by reference in their entireties and for all purposes.

### FIELD

The present disclosure relates to the field of chimeric antigen receptors (CARs) and in particular, to compositions including CARs, and methods of use thereof, such as cancer immunotherapy treatment.

### BACKGROUND

CAR-T cell-based immunotherapies have dramatically improved survival and complete responses in 60-80% of ALL patients and approximately 60% of lymphoma patients. Moreover, CAR-T cell therapies also have shown excellent outcomes in aggressive lymphomas and diffuse large B-cell lymphoma. Overall, in the past few years CAR-T cell therapies have been one of the most exciting and promising therapies for leukemia treatment. Unfortunately, CAR-T cell therapies are associated with severe neurotoxicity, adverse events of 3 and more during clinical trials, and cytokine storm syndrome among others. Therefore, there is a great need to develop strategies that can keep the benefits of CAR therapies and decrease the side-effects.

CAR-T cell therapies are one of the most expensive immunotherapies. Leukemia and lymphoma malignancies are a worldwide problem. Notably, the two CD19-specific CAR-T-cell products currently approved by the United States Food and Drug Administration, are one of the most expensive immunotherapies to date (approximately $500,000 per therapy). Therefore, these therapies can be cost-prohibitive in many parts of the world, and there is a need to develop strategies that can dramatically reduce the costs associated with this immunotherapy for larger impact.

### SUMMARY

Embodiments of the present disclosure include chimeric antigen receptors (CARs) that can be used to target cancer cells regardless of the tumor environment. In accordance with these embodiments, the CARs are administered to a subject along with particles comprising glycolysis accelerating metabolites. In some embodiments, the CARs are engineered to have antigen-specific cytotoxic effects on tumor cells, and in other embodiments, the CARs generate antigen-specific phagocyte-based innate immune responses against cancerous tumors. Phagocyte-based CAR therapies do not require expansion of cells since it takes advantage of large number of phagocytes that are present in the body, and that these cells can then influence the adaptive branch of the immune system, thus saving both time and costs associated with the treatment. The CARs and CAR-based therapies of the present disclosure are believed to be highly desirable for treating cancerous, including but not limited to, solid tumors and diffuse tumors.

In some embodiments, phagocytes, such as neutrophils, monocytes, macrophages and dendritic cells are known to infiltrate solid tumors. Once in the tumor, cancer cells actively prevent the activation of phagocytes, by generating an immunosuppressive microenvironment. Therefore, a therapy that provides delayed activation of phagocytes after they reach the tumor microenvironment, can have a higher chance of killing the cancer cells. Phagocyte-based CAR therapies utilize phagocytosis and NETosis for killing cancer cells.

The CAR-based therapies of the present disclosure include the use of CAR phagocytes that reduce side-effects associated with traditional CAR therapies because of the short lifetime and non-proliferative nature of the activated cells, as well as traditional CARs that induce T-cell mediated cytotoxicity. In some embodiments, delayed activation also provides further protection against side-effects. Specifically, non-activated CAR phagocytes once injected in the body, should get activated after 24 hours, and should be able to infiltrate the tumors, and within one-three days should die, thereby reducing the cytokines released systemically, and this reduces the possibility of cytokine storm syndrome and other severe adverse events associated with CAR therapies.

In accordance with these embodiments, the present disclosure provides a composition comprising: a chimeric antigen receptor (CAR) comprising an extracellular domain comprising a single chain variable fragment (scFv) that binds CD19, CD22, mesothelin, CA-125, or HER2; a cytoplasmic domain comprising a costimulatory domain and a signaling domain; and a glycolysis accelerating metabolite.

In some embodiments, the extracellular domain comprising the scFv and/or the cytoplasmic domain comprising the costimulatory domain and the signaling domain are expressed in an immune cell by delivery of mRNA or plasmid DNA encoding the extracellular domain and/or the cytoplasmic domain.

In some embodiments, the glycolysis accelerating metabolite is in particle form. In some embodiments, the glycolysis accelerating metabolite is in particle form that encapsulates and releases one or more adjuvants in a controlled manner.

In some embodiments, costimulatory domain comprises an intracellular p85-mediated PI3K recruiting domain or a CD3zeta domain. In some embodiments, the signaling domain comprises an FcRgamma, a 4-1BB, a CD28, and/or an ICOS signaling domain.

In some embodiments, the glycolysis accelerating metabolite comprises fructose 6-phosphate (F6P), glucose 6-phosphate (G6P), polyvinylpyrrolidone (PVP), fructose 1,6-biphosphate (F16BP), or succinate.

In some embodiments, the composition is expressed in antigen presenting cells or neutrophils. In some embodiments, the antigen presenting cells are macrophages or dendritic cells.

Embodiments of the present disclosure also include an isolated nucleic acid molecule encoding the CAR in any of the compositions described herein. Embodiments of the present disclosure also include a vector comprising the nucleic acid molecules. Embodiments of the present disclosure also include a cell comprising the nucleic acid molecules or vectors. In some embodiments, the cell is a human antigen presenting cell or human neutrophil. In some embodiments, the human antigen presenting cell is a macrophage. In some embodiments, the cell is a human T cell or an NK cell.

Embodiments of the present disclosure also include an engineered cell comprising any of the compositions described herein. In some embodiments, the engineered cell is an immune cell. In some embodiments, the immune cell is an immune effector cell. In some embodiments, the immune effector cell is a T cell or an NK cell. In some embodiments, the immune effector cell is a macrophage.

Embodiments of the present disclosure also include a pharmaceutical composition comprising a genetically-modified human macrophage comprising a chimeric antigen receptor (CAR) comprising an extracellular domain comprising a single chain variable fragment (scFv) that binds CD19, CD22, mesothelin, CA-125, or HER2, and a cytoplasmic domain comprising a costimulatory domain and a signaling domain; and a glycolysis accelerating metabolite. Embodiments of the present disclosure also include a method for treating a subject suffering from a solid or diffuse tumor comprising introducing into the subject a therapeutically effective amount of the pharmaceutical composition. In some embodiments of the method, the solid or diffuse tumor is a lymphoma and/or leukemia and/or melanoma and/or ovarian tumor.

### BRIEF DESCRIPTION OF THE DRAWINGS

FIG. 1 is a schematic diagram representing various embodiments disclosed herein.

FIG. 2 is a schematic diagram representing various embodiments disclosed herein.

FIGS. 3A-3B provide representative schematics of the CAR constructs for inducing phagocytosis of B cell lymphoma cells. FIG. 3A shows CAR with CD19 single chain variable fragment receptor as the extracellular domain, and intracellular domains of FcRγ with p85 subunit of PI3K recruiting domain, and GFP reporter (tandem construct). FIG. 3B shows extracellular ScFv CD19 domain with intracellular GFP domain (empty construct).

FIGS. 4A-4C provide representative data demonstrating that F16BP can be formulated into phagocytosable particles. Schema of polymer structure is provided in FIG. 4A. Electron microscopy images of F16BP particles are provided in FIG. 4B. Dynamic light scattering demonstrates that the average size of particles is 2±0.3 μm (n=6±stdev) (FIG. 4C).

FIGS. 5A-5B provide representative data demonstrating F16BP microparticles (MPs) upregulate macrophage survival, activation and phagocytic function in a nutrient-poor environment. Macrophages phagocytose larger numbers of beads when F16BP MPs are present (FIG. 5A). F16BP MPs upregulate CD11b expression, frequency of alive cells (ef780), and a marker for activation (CD86) in macrophages in PBS or 10% media (FIG. 5B). N=6±std error. *—p<0.05.

FIG. 6 provides representative data demonstrating that F16BP microparticles (MPs) increase RAW CAR macrophage-like cell survival. N=4±std error. *, $—significantly different, p<0.05.

FIG. 7 provides representative data demonstrating tandem CAR-macs induce death in Ramos lymphoma cells. Representative flow plots and tandem CAR-macs induce higher cell death in Ramos lymphoma than empty CAR-macs for 6 hours (graph at bottom right). N=6±std error. *—p<0.05.

FIG. 8 provides representative data demonstrating the biodistribution of Tandem CAR-macs, with the majority of the cells localized in the tumor. N=4/group±std error.

FIG. 9 provides representative data of fluorescence microscope images of differentiated HL-60 (dHL-60) neutrophil-like cells transfected with Tandem and Empty plasmids are shown (top row). YUMM1.1 melanoma cells and pmaxGFP plasmid were utilized as internal controls (bottom row). It was observed that the Tandem and Empty plasmids can be electroporated in dHL-60 and induce expression of GFP in neutrophil-like cells. Ramos leukemia cells stably expressing RFP are also shown.

FIGS. 10A-10D provide representative data demonstrating that CAR-Neu cells can kill Ramos lymphoma cells. Representative flow plot of a culture of Tandem/Empty CAR-Neu cells and Ramos-RFP cells (FIG. 10A). Representative flow plot shows that ˜89% of CAR-Neu cells that phagocytosed Ramos (GFP+RFP+) are dead. Tandem CAR-Neu cells associate with Ramos cells at significantly higher frequency than empty CAR-Neu (FIG. 10B). Tandem and empty CAR-Neu kill 4 times higher Ramos as compared to non-transfected neutrophils (FIG. 10C). Representative images of transfected neutrophils (expressing GFP) incubated with Ramos lymphoma cell line (expressing RFP) after 6 hours of incubation (FIG. 10D). N=6±stderror; *=p<0.05.

FIG. 11 provides representative data demonstrating that CAR-Neu cells generate NETs when cultured in the presence of Ramos cells. Empty or Tandem CAR transfected dHL60 neutrophils generated NETs (DAPI, arrow). Non-transfected dHL60 are shown as controls.

### DETAILED DESCRIPTION OF SEVERAL EMBODIMENTS

### Terms

The following explanations of terms and methods are provided to better describe the present disclosure and to guide those of ordinary skill in the art in the practice of the present disclosure. The singular terms “a,” “an,” and “the” include plural referents unless context clearly indicates otherwise. Similarly, the word “or” is intended to include “and” unless the context clearly indicates otherwise. The term “comprises” means “includes.” Thus, “comprising A or B,” means “including A, B, or A and B,” without excluding additional elements.

Although methods and materials similar or equivalent to those described herein can be used in the practice or testing of this disclosure, suitable methods and materials are described below.

Unless otherwise explained, all technical and scientific terms used herein have the same meaning as commonly understood by one of ordinary skill in the art to which this disclosure belongs. Definitions of common terms in molecular biology may be found in Benjamin Lewin, Genes V, published by Oxford University Press, 1994 (ISBN 0-19-854287-9); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8).

All publications, patent applications, patents, and other references mentioned herein are incorporated by reference in their entirety. All sequences provided in the disclosed GENBANK Accession numbers are incorporated herein by reference as available on the date of filing this application. In case of conflict, the present specification, including explanations of terms, will control. In addition, the materials, methods, and examples are illustrative only and not intended to be limiting.

In order to facilitate review of the various embodiments of this disclosure, the following explanations of specific terms are provided:

As used herein the term “administration” means to provide or give a subject an agent by any effective route. Exemplary routes of administration include, but are not limited to, injection (such as subcutaneous, intramuscular, intradermal, intraperitoneal, and intravenous), oral, sublingual, rectal, transdermal, intranasal, vaginal and inhalation routes or any combination of techniques thereof.

The disclosed compositions or other therapeutic agents of the present disclosure can be formulated into therapeutically-active pharmaceutical compositions that can be administered to a subject parenterally or orally. Parenteral administration routes include, but are not limited to epidermal, intraarterial, intramuscular (IM, and depot IM), intraperitoneal (IP), intravenous (IV), intrasternal injection or infusion techniques, intranasal (inhalation), intrathecal, injection into the stomach, subcutaneous injections (subcutaneous (SQ and depot SQ), transdermal, topical, and ophthalmic.

The disclosed compositions or other therapeutic agent can be mixed or combined with a suitable pharmaceutically acceptable excipients to prepare pharmaceutical compositions. Pharmaceutically acceptable excipients include, but are not limited to, alumina, aluminum stearate, buffers (such as phosphates), glycine, ion exchangers (such as to help control release of charged substances), lecithin, partial glyceride mixtures of saturated vegetable fatty acids, potassium sorbate, serum proteins (such as human serum albumin), sorbic acid, water, salts or electrolytes such as cellulose-based substances, colloidal silica, disodium hydrogen phosphate, magnesium tri silicate, polyacrylates, polyalkylene glycols, such as polyethylene glycol, polyethylene-polyoxypropylene-block polymers, polyvinyl pyrrolidone, potassium hydrogen phosphate, protamine sulfate, group 1 halide salts such as sodium chloride, sodium carboxymethylcellulose, waxes, wool fat, and zinc salts, for example. Liposomal suspensions may also be suitable as pharmaceutically acceptable carriers.

Upon mixing or addition of a disclosed composition, or other therapeutic agent, the resulting mixture may be a solid, solution, suspension, emulsion, or the like. These may be prepared according to methods known to those of ordinary skill in the art. The form of the resulting mixture depends upon a number of factors, including the intended mode of administration and the solubility of the agent in the selected carrier.

Pharmaceutical carriers suitable for administration of the disclosed compositions or other therapeutic agent include any such carriers known to be suitable for the particular mode of administration. In addition, the disclosed composition or other therapeutic substance can also be mixed with other inactive or active materials that do not impair the desired action, or with materials that supplement the desired action, or have another action.

Methods for solubilizing may be used where the agents exhibit insufficient solubility in a carrier. Such methods are known and include, but are not limited to, dissolution in aqueous sodium bicarbonate, using cosolvents such as dimethylsulfoxide (DMSO), and using surfactants such as TWEEN® (ICI Americas, Inc., Wilmington, Del.).

The disclosed compositions or other therapeutic agent can be prepared with carriers that protect them against rapid elimination from the body, such as coatings or time-release formulations. Such carriers include controlled release formulations, such as, but not limited to, microencapsulated delivery systems. The disclosed compositions or other therapeutic agent is included in the pharmaceutically acceptable carrier in an amount sufficient to exert a therapeutically useful effect, typically in an amount to avoid undesired side effects, on the treated subject. The therapeutically effective concentration may be determined empirically by testing the compounds in known in vitro and in vivo model systems for the treated condition. For example, an acceptable animal model may be used to determine effective amounts or concentrations that can then be translated to other subjects, such as humans, as known in the art.

Injectable solutions or suspensions can be formulated, using suitable non-toxic, parenterally-acceptable diluents or solvents, such as 1,3-butanediol, isotonic sodium chloride solution, mannitol, Ringer's solution, saline solution, or water; or suitable dispersing or wetting and suspending agents, such as sterile, bland, fixed oils, including synthetic mono- or diglycerides, and fatty acids, including oleic acid; a naturally occurring vegetable oil such as coconut oil, cottonseed oil, peanut oil, sesame oil, and the like; glycerine; polyethylene glycol; propylene glycol; or other synthetic solvent; antimicrobial agents such as benzyl alcohol and methyl parabens; antioxidants such as ascorbic acid and sodium bisulfite; buffers such as acetates, citrates, and phosphates; chelating agents such as ethylenediaminetetraacetic acid (EDTA); agents for the adjustment of tonicity such as sodium chloride and dextrose; and combinations thereof. Parenteral preparations can be enclosed in ampoules, disposable syringes, or multiple dose vials made of glass, plastic, or other suitable material. Buffers, preservatives, antioxidants, and the like can be incorporated as required. Where administered intravenously, suitable carriers include physiological saline, phosphate-buffered saline (PBS), and solutions containing thickening and solubilizing agents such as glucose, polyethylene glycol, polypropyleneglycol, and mixtures thereof. Liposomal suspensions, including tissue-targeted liposomes, may also be suitable as pharmaceutically acceptable carriers.

As used herein, the term “agent” is to mean any protein, nucleic acid molecule (including chemically modified nucleic acids), compound, antibody, small molecule, organic compound, inorganic compound, cell, or other molecule of interest. Agent can include a therapeutic agent, a diagnostic agent or a pharmaceutical agent. A therapeutic or pharmaceutical agent is one that alone or together with an additional compound induces the desired response (such as inducing a therapeutic or prophylactic effect when administered to a subject, including treating a subject with or at-risk of acquiring cancer).

In some examples, an agent can act directly or indirectly to alter the activity and/or expression of tumor associated molecule. In a particular example, a therapeutic agent (such as an antisense compound or antibody) significantly alters the expression and/or activity of a tumor associated molecule. An example of a therapeutic agent is one that can decrease the activity of a gene or gene product associated with a tumor, for example as measured by a clinical response (such as an increase survival time or a decrease in one or more signs or symptoms associated with a tumor). Therapeutically agents also include organic or other chemical compounds that mimic the effects of the therapeutically effective peptide, antibody, or nucleic acid molecule.

A “pharmaceutical agent” is a chemical compound or composition capable of inducing a desired therapeutic or prophylactic effect when administered to a subject, alone or in combination with another therapeutic agent(s) or pharmaceutically acceptable carriers. In a particular example, a pharmaceutical agent significantly reduces the expression and/or activity of a tumor-associated molecule thereby increasing a subject's survival time, reducing a sign or symptom associated with the disease, prolonging the onset of tumor signs or symptoms.

The term “antibody,” as used herein, means any antigen-binding molecule or molecular complex comprising at least one complementarity determining region (CDR) that specifically binds to or interacts with a particular antigen. The term “antibody” includes immunoglobulin molecules comprising four polypeptide chains, two heavy (H) chains and two light (L) chains inter-connected by disulfide bonds, as well as multimers thereof (e.g., IgM). The term “antibody” also includes immunoglobulin molecules consisting of four polypeptide chains, two heavy (H) chains and two light (L) chains inter-connected by disulfide bonds. Each heavy chain comprises a heavy chain variable region (abbreviated herein as HCVR or V_{H}) and a heavy chain constant region. The heavy chain constant region comprises three domains, C_{H}1, C_{H}2 and C_{H}3. Each light chain comprises a light chain variable region (abbreviated herein as LCVR or V_{L}) and a light chain constant region. The light chain constant region comprises one domain (C_{L}1). The V_{H} and V_{L} regions can be further subdivided into regions of hypervariability, termed complementarity determining regions (CDRs), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. In different embodiments of the invention, the FRs may be identical to the human germline sequences, or may be naturally or artificially modified. An amino acid consensus sequence may be defined based on a side-by-side analysis of two or more CDRs.

The term “antibody,” as used herein, also includes antigen-binding fragments of full antibody molecules. The terms “antigen-binding portion” of an antibody, “antigen-binding fragment” of an antibody, and the like, as used herein, include any naturally occurring, enzymatically obtainable, synthetic, or genetically engineered polypeptide or glycoprotein that specifically binds an antigen to form a complex. Antigen-binding fragments of an antibody may be derived, e.g., from full antibody molecules using any suitable standard techniques such as proteolytic digestion or recombinant genetic engineering techniques involving the manipulation and expression of DNA encoding antibody variable and optionally constant domains. Such DNA is known and/or is readily available from, e.g., commercial sources, DNA libraries (including, e.g., phage-antibody libraries), or can be synthesized. The DNA may be sequenced and manipulated chemically or by using molecular biology techniques, for example, to arrange one or more variable and/or constant domains into a suitable configuration, or to introduce codons, create cysteine residues, modify, add or delete amino acids, etc.

Non-limiting examples of antigen-binding fragments include: (i) Fab fragments; (ii) F(ab′)2 fragments; (iii) Fd fragments; (iv) Fv fragments; (v) single-chain Fv (scFv) molecules; (vi) dAb fragments; and (vii) minimal recognition units consisting of the amino acid residues that mimic the hypervariable region of an antibody (e.g., an isolated complementarity determining region (CDR) such as a CDR3 peptide), or a constrained FR3-CDR3-FR4 peptide. Other engineered molecules, such as domain-specific antibodies, single domain antibodies, domain-deleted antibodies, chimeric antibodies, CDR-grafted antibodies, diabodies, triabodies, tetrabodies, minibodies, nanobodies (e.g. monovalent nanobodies, bivalent nanobodies, etc.), small modular immunopharmaceuticals (SMIPs), and shark variable IgNAR domains, are also encompassed within the expression “antigen-binding fragment,” as used herein.

An antigen-binding fragment of an antibody will typically comprise at least one variable domain. The variable domain may be of any size or amino acid composition and will generally comprise at least one CDR which is adjacent to or in frame with one or more framework sequences. In antigen-binding fragments having a V_{H} domain associated with a V_{L} domain, the V_{H} and V_{L} domains may be situated relative to one another in any suitable arrangement. For example, the variable region may be dimeric and contain V_{H}-V_{H}, V_{H}-V_{L} or V_{L}-V_{L} dimers. Alternatively, the antigen-binding fragment of an antibody may contain a monomeric V_{H} or V_{L} domain.

In certain embodiments, an antigen-binding fragment of an antibody may contain at least one variable domain covalently linked to at least one constant domain. Non-limiting, exemplary configurations of variable and constant domains that may be found within an antigen-binding fragment of an antibody of the present invention include: (i) V_{H}-C_{H}1; (ii) V_{H}-C_{H}2; (iii) V_{H}-C_{H}3; (iv) V_{H}-C_{H}1-C_{H}2; (v) V_{H}-C_{H}1-C_{H}2-C_{H}3; (vi) V_{H}-C_{H}2-C_{H}3; (vii) V_{H}-C_{L}; (viii) V_{L}-C_{H}1; (ix) V_{L}-C_{H}2; (x) V_{L}-C_{H}3; (xi) V_{L}-C_{H}1-C_{H}2; (xii) V_{L}-C_{H}1-C_{H}2-C_{H}3; (xiii) V_{L}-C_{H}2-C_{H}3; and (xiv) V_{L}-C_{L}. In any configuration of variable and constant domains, including any of the exemplary configurations listed above, the variable and constant domains may be either directly linked to one another or may be linked by a full or partial hinge or linker region. A hinge region may consist of at least 2 (e.g., 5, 10, 15, 20, 40, 60 or more) amino acids which result in a flexible or semi-flexible linkage between adjacent variable and/or constant domains in a single polypeptide molecule. Moreover, an antigen-binding fragment of an antibody of the present invention may comprise a homo-dimer or hetero-dimer (or other multimer) of any of the variable and constant domain configurations listed above in non-covalent association with one another and/or with one or more monomeric V_{H} or V_{L} domain (e.g., by disulfide bond(s)).

In certain embodiments, the antibodies are human antibodies. The term “human antibody”, as used herein, is intended to include antibodies having variable and constant regions derived from human germline immunoglobulin sequences. The human antibodies of the invention may include amino acid residues not encoded by human germline immunoglobulin sequences (e.g., mutations introduced by random or site-specific mutagenesis in vitro or by somatic mutation in vivo), for example in the CDRs and in particular CDR3. However, the term “human antibody”, as used herein, is not intended to include antibodies in which CDR sequences derived from the germline of another mammalian species, such as a mouse, have been grafted onto human framework sequences.

The antibodies may, in some embodiments, be recombinant human antibodies. The term “recombinant human antibody,” as used herein, is intended to include all human antibodies that are prepared, expressed, created or isolated by recombinant means, such as antibodies expressed using a recombinant expression vector transfected into a host cell (described further below), antibodies isolated from a recombinant, combinatorial human antibody library (described further below), antibodies isolated from an animal (e.g., a mouse) that is transgenic for human immunoglobulin genes (see e.g., Taylor et al. (1992) Nucl. Acids Res. 20:6287-6295) or antibodies prepared, expressed, created or isolated by any other means that involves splicing of human immunoglobulin gene sequences to other DNA sequences. Such recombinant human antibodies have variable and constant regions derived from human germline immunoglobulin sequences. In certain embodiments, however, such recombinant human antibodies are subjected to in vitro mutagenesis (or, when an animal transgenic for human Ig sequences is used, in vivo somatic mutagenesis) and thus the amino acid sequences of the V_{H} and V_{L} regions of the recombinant antibodies are sequences that, while derived from and related to human germline V_{H} and V_{L} sequences, may not naturally exist within the human antibody germline repertoire in vivo.

Human antibodies can exist in two forms that are associated with hinge heterogeneity. In one form, an immunoglobulin molecule comprises a stable four chain construct of approximately 150-160 kDa in which the dimers are held together by an interchain heavy chain disulfide bond. In a second form, the dimers are not linked via inter-chain disulfide bonds and a molecule of about 75-80 kDa is formed composed of a covalently coupled light and heavy chain (half-antibody). These forms have been extremely difficult to separate, even after affinity purification.

The frequency of appearance of the second form in various intact IgG isotypes is due to, but not limited to, structural differences associated with the hinge region isotype of the antibody. A single amino acid substitution in the hinge region of the human IgG4 hinge can significantly reduce the appearance of the second form (Angal et al., (1993) Molecular Immunology 30:105) to levels typically observed using a human IgG1 hinge. The instant invention encompasses antibodies having one or more mutations in the hinge, C_{H}2 or C_{H}3 region which may be desirable, for example, in production, to improve the yield of the desired antibody form.

The antibodies may be isolated antibodies. An “isolated antibody,” as used herein, means an antibody that has been identified and separated and/or recovered from at least one component of its natural environment. For example, an antibody that has been separated or removed from at least one component of an organism, or from a tissue or cell in which the antibody naturally exists or is naturally produced, is an “isolated antibody” for purposes of the present invention. An isolated antibody also includes an antibody in situ within a recombinant cell. Isolated antibodies are antibodies that have been subjected to at least one purification or isolation step. According to certain embodiments, an isolated antibody may be substantially free of other cellular material and/or chemicals.

The antibodies disclosed herein may comprise one or more amino acid substitutions, insertions and/or deletions in the framework and/or CDR regions of the heavy and light chain variable domains as compared to the corresponding germline sequences from which the antibodies were derived. Such mutations can be readily ascertained by comparing the amino acid sequences disclosed herein to germline sequences available from, for example, public antibody sequence databases. The present invention includes antibodies, and antigen-binding fragments thereof, which are derived from any of the amino acid sequences disclosed herein, wherein one or more amino acids within one or more framework and/or CDR regions are mutated to the corresponding residue(s) of the germline sequence from which the antibody was derived, or to the corresponding residue(s) of another human germline sequence, or to a conservative amino acid substitution of the corresponding germline residue(s) (such sequence changes are referred to herein collectively as “germline mutations”). A person of ordinary skill in the art, starting with the heavy and light chain variable region sequences disclosed herein, can easily produce numerous antibodies and antigen-binding fragments which comprise one or more individual germline mutations or combinations thereof. In certain embodiments, all of the framework and/or CDR residues within the V_{H} and/or V_{L} domains are mutated back to the residues found in the original germline sequence from which the antibody was derived. In other embodiments, only certain residues are mutated back to the original germline sequence, e.g., only the mutated residues found within the first 8 amino acids of FR1 or within the last 8 amino acids of FR4, or only the mutated residues found within CDR1, CDR2 or CDR3. In other embodiments, one or more of the framework and/or CDR residue(s) are mutated to the corresponding residue(s) of a different germline sequence (i.e., a germline sequence that is different from the germline sequence from which the antibody was originally derived). Furthermore, the antibodies of the present invention may contain any combination of two or more germline mutations within the framework and/or CDR regions, e.g., wherein certain individual residues are mutated to the corresponding residue of a particular germline sequence while certain other residues that differ from the original germline sequence are maintained or are mutated to the corresponding residue of a different germline sequence. Once obtained, antibodies and antigen-binding fragments that contain one or more germline mutations can be easily tested for one or more desired property such as, improved binding specificity, increased binding affinity, improved or enhanced antagonistic or agonistic biological properties (as the case may be), reduced immunogenicity, etc. Antibodies and antigen-binding fragments obtained in this general manner are encompassed within the present invention.

The antibodies may comprise variants of any of the HCVR, LCVR, and/or CDR amino acid sequences disclosed herein having one or more conservative substitutions. For example, the anti-BCMA antibodies may have HCVR, LCVR, and/or CDR amino acid sequences with, e.g., 10 or fewer, 8 or fewer, 6 or fewer, 4 or fewer, etc. conservative amino acid substitutions relative to any of the HCVR, LCVR, and/or CDR amino acid sequences set forth herein.

The term “epitope” refers to an antigenic determinant that interacts with a specific antigen binding site in the variable region of an antibody molecule known as a paratope. A single antigen may have more than one epitope. Thus, different antibodies may bind to different areas on an antigen and may have different biological effects. Epitopes may be either conformational or linear. A conformational epitope is produced by spatially juxtaposed amino acids from different segments of the linear polypeptide chain. A linear epitope is one produced by adjacent amino acid residues in a polypeptide chain. In certain circumstance, an epitope may include moieties of saccharides, phosphoryl groups, or sulfonyl groups on the antigen.

An “autoantibody” is an antibody produced by the immune system that is directed against one or more of the individual's own proteins.

As used herein, the term “antigen presenting cells” means a heterogeneous group of immune cells that mediate the cellular immune response by processing and presenting antigens for recognition by certain lymphocytes, such as T cells. Classical APCs include dendritic cells, macrophages, Langerhans cells and B cells.

The term “substantial identity” or “substantially identical,” when referring to a nucleic acid or fragment thereof, indicates that, when optimally aligned with appropriate nucleotide insertions or deletions with another nucleic acid (or its complementary strand), there is nucleotide sequence identity in at least about 95%, and more preferably at least about 96%, 97%, 98% or 99% of the nucleotide bases, as measured by any well-known algorithm of sequence identity, such as FASTA, BLAST or Gap, as discussed below. A nucleic acid molecule having substantial identity to a reference nucleic acid molecule may, in certain instances, encode a polypeptide having the same or substantially similar amino acid sequence as the polypeptide encoded by the reference nucleic acid molecule.

As applied to polypeptides, the term “substantial similarity” or “substantially similar” means that two peptide sequences, when optimally aligned, such as by the programs GAP or BESTFIT using default gap weights, share at least 95% sequence identity, even more preferably at least 98% or 99% sequence identity. Preferably, residue positions which are not identical differ by conservative amino acid substitutions. A “conservative amino acid substitution” is one in which an amino acid residue is substituted by another amino acid residue having a side chain (R group) with similar chemical properties (e.g., charge or hydrophobicity). In general, a conservative amino acid substitution will not substantially change the functional properties of a protein. In cases where two or more amino acid sequences differ from each other by conservative substitutions, the percent sequence identity or degree of similarity may be adjusted upwards to correct for the conservative nature of the substitution. Means for making this adjustment are well-known to those of skill in the art. See, e.g., Pearson (1994) Methods Mol. Biol. 24: 307-331, herein incorporated by reference. Examples of groups of amino acids that have side chains with similar chemical properties include (1) aliphatic side chains: glycine, alanine, valine, leucine and isoleucine; (2) aliphatic-hydroxyl side chains: serine and threonine; (3) amide-containing side chains: asparagine and glutamine; (4) aromatic side chains: phenylalanine, tyrosine, and tryptophan; (5) basic side chains: lysine, arginine, and histidine; (6) acidic side chains: aspartate and glutamate, and (7) sulfur-containing side chains are cysteine and methionine. Preferred conservative amino acids substitution groups are: valine-leucine-isoleucine, phenylalanine-tyrosine, lysine-arginine, alanine-valine, glutamate-aspartate, and asparagine-glutamine. Alternatively, a conservative replacement is any change having a positive value in the PAM250 log-likelihood matrix disclosed in Gonnet et al., (1992) Science 256: 1443-1445, herein incorporated by reference. A “moderately conservative” replacement is any change having a nonnegative value in the PAM250 log-likelihood matrix.

Sequence similarity for polypeptides, which is also referred to as sequence identity, is typically measured using sequence analysis software. Protein analysis software matches similar sequences using measures of similarity assigned to various substitutions, deletions and other modifications, including conservative amino acid substitutions. For instance, GCG software contains programs such as Gap and Bestfit which can be used with default parameters to determine sequence homology or sequence identity between closely related polypeptides, such as homologous polypeptides from different species of organisms or between a wild type protein and a mutein thereof. See, e.g., GCG Version 6.1. Polypeptide sequences also can be compared using FASTA using default or recommended parameters, a program in GCG Version 6.1. FASTA (e.g., FASTA2 and FASTA3) provides alignments and percent sequence identity of the regions of the best overlap between the query and search sequences (Pearson (2000) supra). Another preferred algorithm when comparing a sequence of the invention to a database containing a large number of sequences from different organisms is the computer program BLAST, especially BLASTP or TBLASTN, using default parameters. See, e.g., Altschul et al., (1990) J. Mol. Biol. 215:403-410 and Altschul et al., (1997) Nucleic Acids Res. 25:3389-402, each herein incorporated by reference.

As used herein, the terms “nucleic acid” or “polynucleotides” refers to nucleotides and/or polynucleotides, such as deoxyribonucleic acid (DNA) or ribonucleic acid (RNA), oligonucleotides, fragments generated by the polymerase chain reaction (PCR), and fragments generated by any of ligation, scission, endonuclease action, and exonuclease action. Nucleic acid molecules can be composed of monomers that are naturally-occurring nucleotides (such as DNA and RNA), or analogs of naturally-occurring nucleotides (e.g., enantiomeric forms of naturally-occurring nucleotides), or a combination of both. Modified nucleotides can have alterations in sugar moieties and/or in pyrimidine or purine base moieties. Sugar modifications include, for example, replacement of one or more hydroxyl groups with halogens, alkyl groups, amines, and azido groups, or sugars can be functionalized as ethers or esters. Moreover, the entire sugar moiety can be replaced with sterically and electronically similar structures, such as aza-sugars and carbocyclic sugar analogs. Examples of modifications in a base moiety include alkylated purines and pyrimidines, acylated purines or pyrimidines, or other well-known heterocyclic substitutes. Nucleic acid monomers can be linked by phosphodiester bonds or analogs of such linkages. Nucleic acids can be either single stranded or double stranded.

The term “chimeric antigen receptor” (CAR) refers to molecules that combine a binding domain against a component present on the target cell, for example an antibody-based specificity for a desired antigen (e.g., a tumor antigen) with one or more macrophage-activating intracellular domains to generate a chimeric protein that exhibits a specific anti-target cellular immune activity. Generally, as used herein, CARs include an extracellular single chain antibody-binding domain (scFv) fused to the one or more macrophage intracellular signaling domain, and have the ability, when expressed in cells, to redirect antigen recognition based on the monoclonal antibody's specificity. CARs of the present disclosure also include an extracellular single chain antibody-binding domain (scFv) fused to transmembrane and intracellular signaling domains that can induce T-cell and NK-cell mediated cytotoxicity in a target cancer cell (e.g., any of first through fifth generation CARs).

The term “vector,” as used herein, includes, but is not limited to, a viral vector, a plasmid, an RNA vector or a linear or circular DNA or RNA molecule that may consists of chromosomal, non-chromosomal, semi-synthetic or synthetic nucleic acids. In some cases, the vectors are those capable of autonomous replication (episomal vector) and/or expression of nucleic acids to which they are linked (expression vectors). Large numbers of suitable vectors are known to those of skill in the art and are commercially available. Viral vectors include retrovirus, adenovirus, parvovirus (e.g., adenoassociated viruses), coronavirus, negative strand RNA viruses such as orthomyxovirus (e.g., influenza virus), rhabdovirus (e.g., rabies and vesicular stomatitis virus), paramyxovirus (e.g. measles and Sendai), positive strand RNA viruses such as picornavirus and alphavirus, and double-stranded DNA viruses including adenovirus, herpesvirus (e.g., Herpes Simplex virus types 1 and 2, Epstein-Barr virus, cytomegalovirus), and poxvirus (e.g., vaccinia, fowlpox and canarypox). Other viruses include Norwalk virus, togavirus, flavivirus, reoviruses, papovavirus, hepadnavirus, and hepatitis virus, for example. Examples of retroviruses include: avian leukosis-sarcoma, mammalian C-type, B-type viruses, D type viruses, HTLV-BLV group, and lentivirus.

A “costimulatory domain” or “costimulatory molecule” refers to the cognate binding partner on a cell that specifically binds with a costimulatory ligand, thereby mediating a costimulatory response by the cell, such as, but not limited to proliferation. Costimulatory molecules include, but are not limited to, an MEW class I molecule, BTLA and Toll ligand receptor. Examples of costimulatory molecules include CD27, CD28, CD8, 4-1BB, CD137, OX40, CD30, CD40, PD-1, ICOS, lymphocyte function-associated antigen-1 (LFA-1), CD2, CD7, LIGHT, NKG2C, B7-H3 and a ligand that specifically binds with CD83 and the like. A costimulatory molecule is a cell surface molecule other than an antigen receptor or their ligands that is required for an efficient immune response.

A “costimulatory ligand” refers to a molecule on an antigen presenting cell that specifically binds a cognate costimulatory molecule on the cell, thereby providing a signal which, in addition to the primary signal provided by, for instance, binding of a TCR/CD3 complex with an MHC molecule loaded with peptide, mediates a cell response, including, but not limited to, proliferation activation, differentiation and the like. A costimulatory ligand can include but is not limited to CD7, B7-1 (CD80), B7-2 (CD86), PD-L1, PD-L2, 4-1BBL, OX40L, inducible costimulatory ligand (ICOS-L), intercellular adhesion molecule (ICAM), CD30L, CD40, CD70, CD83, HLA-G, MICA, M1CB, HVEM, lymphotoxin beta receptor, 3/TR6, ILT3, ILT4, an agonist or antibody that binds Toll ligand receptor and a ligand that specifically binds with B7-H3.

A “costimulatory signal” refers to a signal, which in combination with a primary signal, such as TCR/CD3 ligation, leads to T cell proliferation and/or upregulation or downregulation of key molecules.

The term “extracellular ligand-binding domain,” as used herein, refers to an oligo- or polypeptide that is capable of binding a ligand, e.g., a cell surface molecule. For example, the extracellular ligand-binding domain may be chosen to recognize a ligand that acts as a cell surface marker on target cells associated with a particular disease state (e.g., cancer). Examples of cell surface markers that may act as ligands include those associated with viral, bacterial and parasitic infections, autoimmune disease and cancer cells.

The term “subject” or “patient” as used herein includes all members of the animal kingdom including non-human primates and humans. In one embodiment, patients are humans with a cancer.

A “signal transducing domain” or “signaling domain” of a CAR, as used herein, is responsible for intracellular signaling following the binding of an extracellular ligand binding domain to the target resulting in the activation of the immune cell and immune response. In other words, the signal transducing domain is responsible for the activation of at least one of the normal effector functions of the immune cell in which the CAR is expressed. Thus, the term “signal transducing domain” refers to the portion of a protein which transduces the effector function signal and directs the cell to perform a specialized function. Examples of signal transducing domains for use in a CAR can be the cytoplasmic sequences of a cell receptor and co-receptors that act in concert to initiate signal transduction following antigen receptor engagement, as well as any derivate or variant of these sequences and any synthetic sequence that has the same functional capability. In some cases, signaling domains comprise two distinct classes of cytoplasmic signaling sequences, those that initiate antigen-dependent primary activation, and those that act in an antigen-independent manner to provide a secondary or co-stimulatory signal. Primary cytoplasmic signaling sequences can comprise signaling motifs which are known as immunoreceptor tyrosine-based activation motifs of ITAMs. ITAMs are well defined signaling motifs found in the intracytoplasmic tail of a variety of receptors that serve as binding sites for syk/zap70 class tyrosine kinases. Exemplary ITAMs include those derived from TCRzeta, FcRgamma, FcRbeta, FcRepsilon, CD3gamma, CD3delta, CD3epsilon, CD5, CD22, CD79a, CD79b and CD66d.

The term “alteration or modulation in expression” as used herein is an alteration in expression of a gene, gene product or modulator thereof, such as one or more tumor associated molecules disclosed herein, refers to a change or difference, such as an increase or decrease, in the level of the gene, gene product, or modulators thereof that is detectable in a biological sample (such as a sample from a subject at-risk or having a tumor) relative to a control (such as a sample from a subject without a tumor) or a reference value known to be indicative of the level of the gene, gene product or modulator thereof in the absence of the disease. An “alteration” in expression includes an increase in expression (up-regulation) or a decrease in expression (down-regulation).

As used herein, the term “binding or stable binding” is an association between two substances or molecules, such as the hybridization of one nucleic acid molecule to another (or itself), the association of an antibody with a peptide, or the association of a protein with another protein or nucleic acid molecule. An oligonucleotide molecule binds or stably binds to a target nucleic acid molecule if a sufficient amount of the oligonucleotide molecule forms base pairs or is hybridized to its target nucleic acid molecule, to permit detection of that binding. “Preferentially binds” indicates that one molecule binds to another with high affinity, and binds to heterologous molecules at a low affinity.

Binding can be detected by any procedure known to one skilled in the art, such as by physical or functional properties of the target complex. For example, binding can be detected functionally by determining whether binding has an observable effect upon a biosynthetic process such as expression of a gene, DNA replication, transcription, translation, and the like. Methods of detecting binding of an antibody to a protein are disclosed herein and also can include known methods of protein detection, such as Western blotting.

As used herein, the term “clinical outcome” refers to the health status of a patient following treatment for a disease or disorder, such as cancer or in the absence of treatment. Clinical outcomes include, but are not limited to, an increase in the length of time until death, a decrease in the length of time until death, an increase in the chance of survival, an increase in the risk of death, survival, disease-free survival, chronic disease, metastasis, advanced or aggressive disease, disease recurrence, death, and favorable or poor response to therapy.

As used herein, the term “contacting” is the placement in direct physical association, including both a solid and liquid form. Contacting an agent with a cell can occur in vitro by adding the agent to isolated cells or in vivo by administering the agent to a subject.

As used herein, the term “control” is a sample or standard used for comparison with a test sample, such as a biological sample obtained from a patient (or plurality of patients) without a particular disease or condition, such as cancer. In some embodiments, the control is a sample obtained from a healthy patient (or plurality of patients) (also referred to herein as a “normal” control), such as a normal biological sample or from a non-cancerous biological sample from the patient that has particular disease or condition, such as cancer. In some embodiments, the control is a historical control or standard value (e.g., a previously tested control sample or group of samples that represent baseline or normal values (e.g., expression values), such as baseline or normal values of a particular gene, gene product in a subject without cancer). In some examples, the control is a standard value representing the average value (or average range of values) obtained from a plurality of patient samples (such as an average value or range of values of the gene or gene products in the subjects without cancer).

As used herein, the term “decrease” is to reduce the quality, amount, or strength of something. In one example, a therapy decreases one or more symptoms associated with a particular condition or disease, for example as compared to the response in the absence of the therapy.

Examples of processes that decrease transcription include those that facilitate degradation of a transcription initiation complex, those that decrease transcription initiation rate, those that decrease transcription elongation rate, those that decrease processivity of transcription and those that increase transcriptional repression. Gene downregulation can include reduction of expression above an existing level. Examples of processes that decrease translation include those that decrease translational initiation, those that decrease translational elongation and those that decrease mRNA stability.

Gene downregulation includes any detectable decrease in the production of a gene product. In certain examples, production of a gene product decreases by at least 2-fold, for example at least 3-fold or at least 4-fold, as compared to a control (such an amount of gene expression in a normal cell). In one example, a control is a relative amount of gene expression or protein expression in a biological sample taken from a subject who does not have cancer. Such decreases can be measured using the methods disclosed herein. For example, “detecting or measuring expression of a gene product” includes quantifying the amount of the gene, gene product or modulator thereof present in a sample. Quantification can be either numerical or relative. Detecting expression of the gene, gene product or modulators thereof can be achieved using any method known in the art or described herein, such as by measuring nucleic acids by PCR (such as RT-PCR) and proteins by ELISA. In primary embodiments, the change detected is an increase or decrease in expression as compared to a control, such as a reference value or a healthy control subject. In some examples, the detected increase or decrease is an increase or decrease of at least two-fold compared with the control or standard. Controls or standards for comparison to a sample, for the determination of differential expression, include samples believed to be normal (in that they are not altered for the desired characteristic, for example a sample from a subject who does not have cancer) as well as laboratory values (e.g., range of values), even though possibly arbitrarily set, keeping in mind that such values can vary from laboratory to laboratory.

Laboratory standards and values can be set based on a known or determined population value and can be supplied in the format of a graph or table that permits comparison of measured, experimentally determined values.

The level of expression in either a qualitative or quantitative manner can detect nucleic acid or protein. Exemplary methods include microarray analysis, RT-PCR, Northern blot, Western blot, and mass spectrometry.

As used herein, the term “detecting” means identifying the presence, absence or relative or absolute amount of the object to be detected.

As used herein, the term “effective amount” is an amount of agent that is sufficient to generate a desired response, such as reducing lessening, ameliorating, eliminating, preventing, or inhibiting one or more signs or symptoms associated with a condition or disease treated and may be empirically determined. When administered to a subject, a dosage will generally be used that will achieve target tissue/cell concentrations. In some examples, an “effective amount” is one that treats one or more symptoms and/or underlying causes of any of a disorder or disease. In some examples, an “effective amount” is a therapeutically effective amount in which the agent alone with an additional therapeutic agent(s) (for example anti-cancer agents), induces the desired response such as treatment of a particular type of cancer.

In particular examples, it is an amount of an agent capable of modulating one or more of the disclosed genes, gene products or modulators thereof associated with a particular condition or disease by least 20%, at least 50%, at least 60%, at least 70%, at least 80%, at least 90%, at least 95%, at least 98%, or even at least 100% (elimination of the disease to a point beyond detection) by the agent.

In some examples, an effective amount is an amount of a pharmaceutical preparation that alone, or together with a pharmaceutically acceptable carrier or one or more additional therapeutic agents, induces the desired response.

In one example, a desired response is to increase the subject's survival time by slowing the progression of the disease. The disease does not need to be completely inhibited for the pharmaceutical preparation to be effective. For example, a pharmaceutical preparation can decrease the progression of the disease by a desired amount, for example by at least 20%, at least 50%, at least 60%, at least 70%, at least 80%, at least 90%, at least 95%, at least 98%, or even at least 100%, as compared to the progression typical in the absence of the pharmaceutical preparation.

In another or additional example, it is an amount sufficient to partially or completely alleviate symptoms of the condition/disease within the subject. Treatment can involve only slowing the progression of the disease temporarily, but can also include halting or reversing the progression of the disease permanently.

Effective amounts of the agents described herein can be determined in many different ways, such as assaying for a reduction in of one or more signs or symptoms associated with the disease/condition in the subject or measuring the expression level of one or more molecules known to be associated with the disease/condition. Effective amounts also can be determined through various in vitro, in vivo or in situ assays, including the assays described herein.

The disclosed therapeutic agents can be administered in a single dose, or in several doses, for example daily, during a course of treatment. However, the effective amount can be dependent on the source applied (for example a nucleic acid molecule isolated from a cellular extract versus a chemically synthesized and purified nucleic acid), the subject being treated, the severity and type of the condition being treated, and the manner of administration.

As used herein, the phrase “inhibiting a disease or condition” is a phrase referring to inhibiting the development of a disease or condition, such as reducing, decreasing or delaying a sign or symptom associated with the disease or condition, for example, in a subject who is at-risk of acquiring the disease/condition or has the particular disease/condition. Particular methods of the present disclosure provide methods for inhibiting or reducing cancer.

As used herein, an “isolated” biological component (such as a nucleic acid molecule, protein, or cell) is a component that has been substantially separated or purified away from other biological components in the cell of the organism, or the organism itself, in which the component naturally occurs, such as other chromosomal and extra-chromosomal DNA and RNA, proteins and cells. Nucleic acid molecules and proteins that have been “isolated” include nucleic acid molecules and proteins purified by standard purification methods. The term also embraces nucleic acid molecules and proteins prepared by recombinant expression in a host cell as well as chemically synthesized nucleic acid molecules and proteins.

As used herein “Label or Detectable Moiety” is a composition detectable by spectroscopic, photochemical, biochemical, immunochemical, electromagnetic, or chemical means. For example, useful labels include radiolabels such as ³²P, ³⁵S, or ¹²⁵I; heavy isotopes such as ¹⁵N or ¹³C or heavy atoms such as selenium or metals; fluorescent dyes; chromophores, electron-dense reagents; enzymes that generate a detectable signal (e.g., alkaline phosphatase or peroxidase, as commonly used in an ELISA); or spin labels. The label or detectable moiety has or generates a measurable signal, such as a radioactive, chromogenic, or fluorescent signal, that can be used to quantify the amount of bound detectable moiety in a sample. The detectable moiety can be incorporated in or attached to a molecule (such as a protein, for example, an antibody) either covalently, or through ionic, van der Waals or hydrogen bonds, e.g., or by incorporation of labeled precursors. The label or detectable moiety may be directly or indirectly detectable. Indirect detection can involve the binding of a second directly or indirectly detectable moiety to the detectable moiety. For example, the detectable moiety can be the ligand of a binding partner, such as biotin, which is a binding partner for streptavidin, which can be linked to a directly detectable label. The binding partner may itself be directly detectable, for example, an antibody may be itself labeled with a fluorescent molecule. The binding partner also may be indirectly detectable, for example, it may be bound by another moiety that comprises a label. Quantitation of the signal is achieved by any appropriate means, e.g., fluorescence detection, spectrophotometric detection (e.g., absorption at a particular wavelength), scintillation counting, mass spectrometry, densitometry, or flow cytometry. Methods for labeling and guidance in the choice of labels appropriate for various purposes are discussed for example in Sambrook et al. (Molecular Cloning: A Laboratory Manual, Cold Spring Harbor, N.Y., 1989) and Ausubel et al., (In Current Protocols in Molecular Biology, John Wiley & Sons, New York, 1998). In particular examples, a label or detectable moiety is conjugated to a binding agent that specifically binds to one or more of the tumor-associated molecules.

The term “cytotoxic agent” as used herein refers to a substance that inhibits or prevents the function of cells and/or causes destruction of cells. The term is intended to include radioactive isotopes (e.g., At²¹¹, I¹³¹, I¹²⁵, Y⁹⁰, Re¹⁸⁶, Re¹⁸⁸, Sm¹⁵³, Bi²¹², P³² and radioactive isotopes of Lu), chemotherapeutic agents e.g., methotrexate, adriamicin, vinca alkaloids (vincristine, vinblastine, etoposide), doxorubicin, melphalan, mitomycin C, chlorambucil, daunorubicin or other intercalating agents, enzymes and fragments thereof such as nucleolytic enzymes, antibiotics, and toxins such as small molecule toxins or enzymatically active toxins of bacterial, fungal, plant or animal origin, including fragments and/or variants thereof, and the various antitumor or anticancer agents disclosed below. Other cytotoxic agents are described below. A tumoricidal agent causes destruction of tumor cells.

As used herein, “prognosis” is a prediction of the course of a disease, such as cancer. The prediction can include determining the likelihood of a subject to develop aggressive, recurrent disease, to survive a particular amount of time (e.g., determine the likelihood that a subject will survive 1, 2, 3 or 5 years), to respond to a particular therapy or combinations thereof.

As used herein, sample (or biological sample) is a biological specimen containing genomic DNA, RNA (including mRNA), protein, cells (such as neutrophil-cells) or combinations thereof, obtained from a subject. Examples include, but are not limited to, peripheral blood, urine, saliva, tissue biopsy, surgical specimen, and autopsy material.

As used herein, the term “sensitivity” is the percent of diseased individuals (individuals with prostate cancer) in which the biomarker of interest is detected (true positive number/total number of diseased×100). Non-diseased individuals diagnosed by the test as diseased are “false positives”.

As used herein, the term “specificity” is the percent of non-diseased individuals for which the biomarker of interest is not detected (true negative/total number without disease×100). Diseased individuals not detected by the assay are “false negatives.” Subjects who are not diseased and who test negative in the assay, are termed “true negatives.”

As used herein, the term “signs or symptoms” means any subjective evidence of disease or of a subject's condition, e.g., such evidence as perceived by the subject; a noticeable change in a subject's condition indicative of some bodily or mental state. A “sign” is any abnormality indicative of disease, discoverable on examination or assessment of a subject. A sign is generally an objective indication of disease.

As used herein, a “standard” is a substance or solution of a substance of known amount, purity or concentration. A standard can be compared (such as by spectrometric, chromatographic, or spectrophotometric analysis) to an unknown sample (of the same or similar substance) to determine the presence of the substance in the sample and/or determine the amount, purity or concentration of the unknown sample. In one embodiment, a standard is a peptide standard. An internal standard is a compound that is added in a known amount to a sample prior to sample preparation and/or analysis and serves as a reference for calculating the concentrations of the components of the sample. In one example, nucleic acid standards serve as reference values for tumor or non-tumor expression levels of specific nucleic acids. In some examples, peptide standards serve as reference values for tumor or non-tumor expression levels of specific peptides. Isotopically-labeled peptides are particularly useful as internal standards for peptide analysis since the chemical properties of the labeled peptide standards are almost identical to their non-labeled counterparts. Thus, during chemical sample preparation steps (such as chromatography, for example, HPLC) any loss of the non-labeled peptides is reflected in a similar loss of the labeled peptides.

As used herein, the term “tissue” is a plurality of functionally related cells. A tissue can be a suspension, a semi-solid, or solid. Tissue includes cells collected from a subject.

As used herein, the phrase “treating a disease” is therapeutic intervention that ameliorates a sign or symptom of a disease or pathological condition related to cancer, such as a sign or symptom of cancer, or a specific type of cancer. Treatment can induce remission or cure of a condition or slow progression, for example, in some instances can include inhibiting the full development of a disease, for example preventing development of cancer. Prevention of a disease does not require a total absence of disease. For example, a decrease of at least 10%, such as at least 20%, at least 25%, at least 30%, at least 40%, at least 50%, decrease in a sign or symptom associated with the condition or disease can be sufficient.

As used herein, “CD19” is a transmembrane protein that in humans is encoded by the gene CD19. In humans, CD19 is expressed in all B lineage cells. CD19 plays two major roles in human B cells. It acts as an adaptor protein to recruit cytoplasmic signaling proteins to the membrane and it works within the CD19/CD21 complex to decrease the threshold for B cell receptor signaling pathways. Due to its presence on all B cells, it is a biomarker for B lymphocyte development, lymphoma diagnosis and can be utilized as a target for leukemia immunotherapies. The term “anti-CD19 domain” is a domain capable of binding to CD19 expressed in a B-cell and inhibiting CD19 activities. The sequence of CD19 is known to those of skill in the art, see for example, Gene ID: 930, on the NCBI website updated on updated on Jul. 29, 2020, which is hereby incorporated by reference.

As used herein, the “FcRgamma” is an adapter protein associated with a wide spectrum of receptors in a variety of innate immune cells to mediate intracellular signaling pathways when their cognate receptor is engaged. These adapter proteins are coupled to their receptors through charged residues within the transmembrane regions of the adapter and receptor. FcRgamma contains specific protein domains (referred to as immunoreceptor tyrosine-based activation motifs) that serve as the substrates and docking sites for kinases, allowing amplification of intracellular signaling reactions. FcRgamma is capable of modulating innate immune responses.

As used herein, the term “p85” refers to the regulatory unit of phosphoinositide 3-kinases (PI3Ks). p85 is composed of an SH3 domain, a RhoGap domain, a N-terminal SH2 (nSH2) domain, a inter SH2 (iSH2) domain, and C-terminal (cSH2) domain. There are two inhibitory interactions between p110alpha and p85 of P13K: 1) p85 nSH2 domain with the C2, helical, and kinase domains of p110alpha and 2) p85 iSH2 domain with C2 domain of p110alpha. There are three inhibitory interactions between p110beta and p85 of P13K: 1) p85 nSH2 domain with the C2, helical, and kinase domains of p110beta, 2) p85 iSH2 domain with C2 domain of p110beta, and 3) p85 cSH2 domain with the kinase domain of p110beta.

As used herein, the term “F16BP or (fructose 1,6-biphosphate)” is fructose sugar phosphorylated on carbons 1 and 6 (i.e., is a fructosephosphate). The β-D-form of this compound is common in cells. Upon entering the cell, most glucose and fructose is converted to fructose 1,6-bisphosphate. F16BP is a glycolysis accelerating metabolite. Additional glycolysis accelerating metabolites include, but are not limited to, fructose 6-phosphate (F6P), glucose 6-phosphate (G6P), polyvinylpyrrolidone (PVP), and succinate.

As used herein, “phagocytes” are a type of white blood cell that use phagocytosis to engulf bacteria, foreign particles, and dying cells to protect the body. They bind to pathogens and internalize them in a phagosome, which acidifies and fuses with lysosomes in order to destroy the contents. They are a key component of the innate immune system. Phagocytes can include neutrophils, monocytes, macrophages, granulocytes and dendritic cells. Phagocytes are capable of infiltrating solid tumors.

As used herein, CD22 or cluster of differentiation-22, is a molecule belonging to the SIGLEC family of lectins. It is found on the surface of mature B cells and to a lesser extent on some immature B cells. Generally speaking, CD22 is a regulatory molecule that prevents the overactivation of the immune system and the development of autoimmune diseases. CD22 is a sugar binding transmembrane protein, which specifically binds sialic acid with an immunoglobulin (Ig) domain located at its N-terminus. The presence of Ig domains makes CD22 a member of the immunoglobulin superfamily. CD22 functions as an inhibitory receptor for B cell receptor (BCR) signaling. It is also involved in the B cell trafficking to Peyer's patches in mice. The sequence of CD22 is known to those of skill in the art, see for example, Gene ID: 933, on the NCBI website updated on updated on Jun. 7, 2020, which is hereby incorporated by reference.

As used herein, mesothelin (MSLN) is a tumor-associated antigen broadly overexpressed on various malignant tumor cells, while its expression is generally limited to normal mesothelial cells. The MSLN gene encodes a 71-KD precursor, which is a glycosylphosphatidylinositol (GPI)-anchored membrane glycoprotein that is cleaved into two products at arginine 295 (Arg295): a soluble 31-KD N-terminal protein called megakaryocyte potentiating factor (MPF) and a 40-KD membrane-bound fragment called MSLN (mesothelin). Both MPF and MSLN are bioactive, but their exact functions remain unclear. MPF was initially reported to stimulate megakaryocyte colony formation in the presence of interleukin-3 in mice but not alone, while its activity is unknown in humans. MSLN was first described as a membrane protein expressed on mesothelioma and ovarian cancer cells and normal mesothelial cells. A previous study showed that MSLN seemed to be a nonessential component in normal cells, as MSLN knockout mice did not present with abnormal development or reproduction. In contrast, preclinical and clinical studies showed that aberrant MSLN expression on tumor cells plays an important role in promoting proliferation and invasion. MSLN has also been identified as a receptor of CA125 that mediates cell adhesion. The interaction of CA125 and MSLN play an important role in ovarian cancer cell peritoneal implantation and increase the motility and invasion of pancreatic carcinoma cells. The overexpression of MSLN could activate the NFκB, MAPK, and PI3K pathways and subsequently induce resistance to apoptosis or promote cell proliferation, migration, and metastasis by inducing the activation and expression of MMP7 and MMP9. An increase in tumor burden and poor overall survival are associated with elevated MSLN expression according to clinical observations. The sequence of mesothelin is known to those of skill in the art, see for example, Gene ID: 10232, on the NCBI website updated on updated on Jun. 7, 2020, which is hereby incorporated by reference.

As used herein, CA-125 (MCU16) encodes a protein that is a member of the mucin family. Mucins are high molecular weight, O-glycosylated proteins that play an important role in forming a protective mucous barrier, and are found on the apical surfaces of the epithelia. The encoded protein is a membrane-tethered mucin that contains an extracellular domain at its amino terminus, a large tandem repeat domain, and a transmembrane domain with a short cytoplasmic domain. The amino terminus is highly glycosylated, while the repeat region contains 156 amino acid repeats unit that are rich in serines, threonines, and prolines. Interspersed within the repeats are Sea urchin sperm protein Enterokinase and Agrin (SEA) modules, leucine-rich repeats and ankyrin (ANK) repeats. These regions together form the ectodomain, and there is a potential cleavage site found near an SEA module close to the transmembrane domain. This protein is thought to play a role in forming a barrier, protecting epithelial cells from pathogens. Products of this gene have been used as a marker for different cancers, with higher expression levels associated with poorer outcomes. The sequence of CA-125 is known to those of skill in the art, see for example, Gene ID: 94025, on the NCBI website updated on updated on Jun. 7, 2020, which is hereby incorporated by reference.

As used herein, HER 2 is Receptor tyrosine-protein kinase erbB-2, also known as CD340 (cluster of differentiation 340), proto-oncogene Neu, Erbb2 (rodent), or ERBB2 (human), is a protein that in humans is encoded by the ERBB2 gene. ERBB is abbreviated from erythroblastic oncogene B, a gene isolated from avian genome. It is also frequently called HER2 (from human epidermal growth factor receptor 2) or HER2/neu. HER2 is a member of the human epidermal growth factor receptor (HER/EGFR/ERBB) family. Amplification or over-expression of this oncogene has been shown to play a role in the development and progression of certain aggressive types of breast cancer. In recent years, the protein has become an important biomarker and target of therapy for approximately 30% of breast cancer patients. The sequence of HER2 is known to those of skill in the art, see for example, Gene ID: 2064, on the NCBI website updated on Jul. 22, 2020, which is hereby incorporated by reference.

### Compositions and Methods of Use

Chimeric antigen receptor (CAR)-T cell treatments (pre-clinical and clinical) have revolutionized cancer immunotherapies. However, efficient, non-viral, less toxic and cost-effective methods are desirable for transfecting immune cells for CAR therapy. Disclosed herein are CAR-based neutrophils (CAR-Neu) and CAR-based macrophages (CAR-macs) and CAR-dendritic cells (CAR-DCs), collectively called CAR-antigen presenting cells or CAR-phagocytes that overcome the limitations associated with CAR-T cell therapies. CAR-phagocytes have several advantages over CAR-T cells. Specifically, collectively phagocytes are the most abundant immune cells present in the blood, (greater than 5×10⁹/Liter, approximately 75% of all white blood cells). Phagocyte numbers are elevated in the blood during several types of cancers. They are capable of infiltrating the tumor and inducing inflammation in tumor microenvironment via release of reactive oxygen species (ROS). Therefore, phagocytes do not need to be expanded like CAR-T cells, and can save both time and expense associated with CAR therapies. Furthermore, phagocytes once activated cannot undergo clonal expansion (lifetime less than 1-3 days) which can alleviate the duration/possibility of cytokine storm events, which are a major safety concern in CAR-T cell therapies. The disclosed therapies pertain to delivering CAR plasmids to phagocytes and inducing tumor regression. Embodiments include ex vivo electroporation, and in vivo lipopolymer based targeting and transfection. Embodiments utilize a non-viral macrophage transfection strategy to generate CAR-Macs or CAR-neutrophils or CAR-dendritic cells. In embodiments, the transfected CAR-Macs or CAR-DCs phagocytose glycolysis accelerating metabolites in the micro/nanoparticle format, and survive in nutrient-poor tumor microenvironment, and are able to infiltrate the solid lymphoma tumors and provide tumor regression.

In some embodiments, the present disclosure also includes CARs comprising an extracellular target-binding domain, a hinge region, a transmembrane domain that anchors the CAR to the cell membrane, and one or more intracellular domains that transmit activation signals. Depending on the number of costimulatory domains, CARs can be classified into first (CD3zeta only), second (one costimulatory domain+CD3zeta), or third generation CARs (more than one costimulatory domain+CD3zeta). Introduction of CAR polypeptides into a T cell or an NK cell redirects the T cell with additional antigen specificity and provides the necessary signals to drive full T cell activation. Because antigen recognition by CAR T cells and NK cells is based on the binding of the target-binding single-chain variable fragment (scFv) to intact surface antigens, targeting of tumor cells is not MEW restricted, co-receptor dependent, or dependent on processing and effective presentation of target epitopes.

In accordance with the above embodiments, compositions of the present disclosure include particles based on fructose, 1,6 biphosphate (F16BP—rate limiting glycolysis step), with or without poly (I:C) (adjuvant activating macrophages) as the backbone. These particles can be phagocytosed by phagocytes, such as human macrophages and human Dendritic cells differentiated from monocytes of human blood.

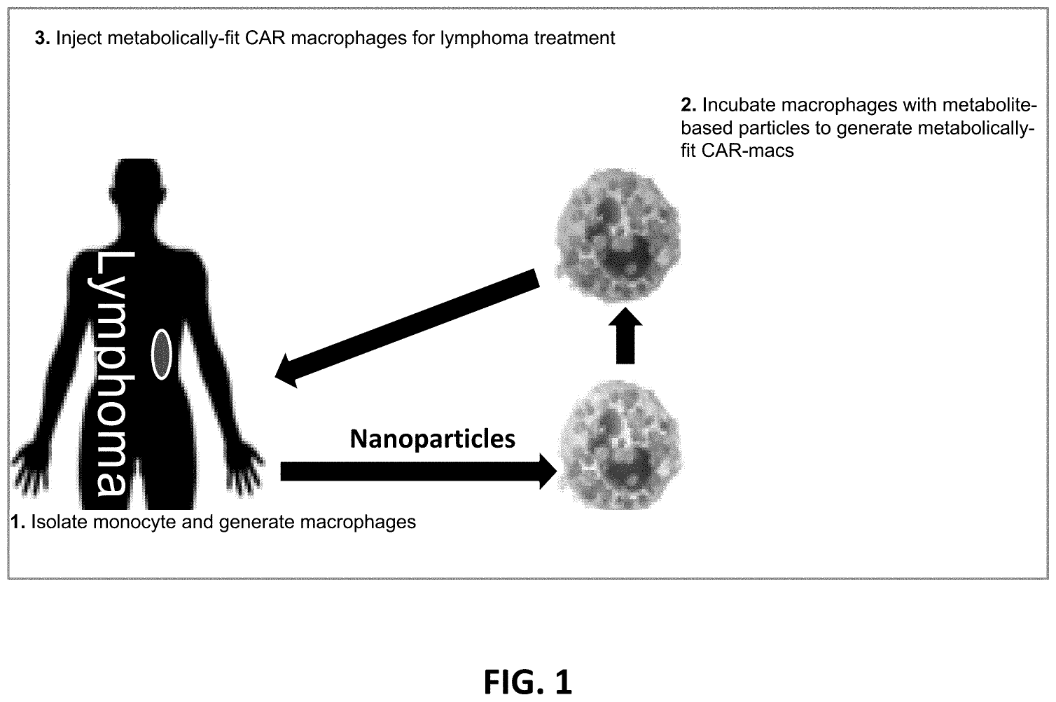

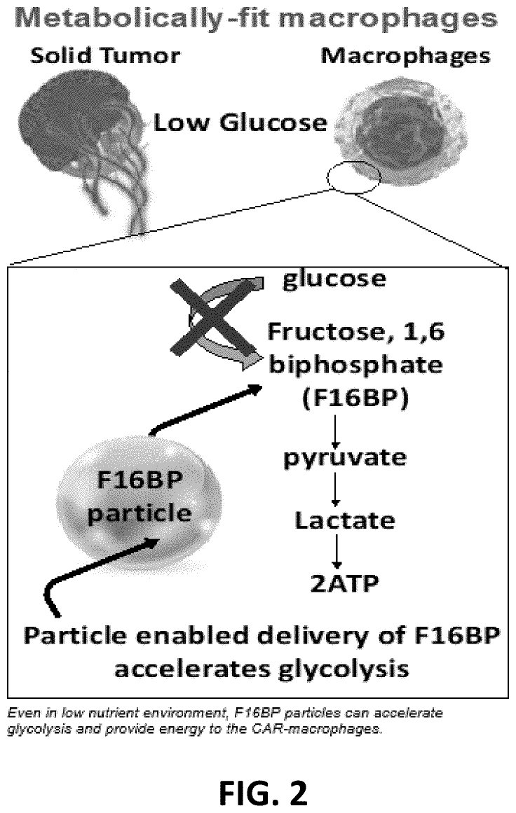

In some embodiments, plasmids encoding CAR expression of extracellular human CD19 single chain variable fragment and intracellular p85-mediated PI3K recruiting (phagocytic/activation pathway in macrophages) domain are disclosed. In embodiments, disclosed plasmids can be transfected into cells, such as neutrophils or macrophage cells to form CAR-macrophage cells with phagocytic activities. In embodiments, CAR-macrophage cells are provided to a subject in need thereof, such as by intravenous injections. FIG. 1 provides a schematic of this exemplary method. FIG. 2 and as disclosed herein illustrate how metabolically-fit CAR macrophages can be used to kill cancer cells, such as lymphoma cells by phagocytosis. In embodiments, the metabolite, F16BP is delivered to CAR macrophages to maintain the activation state of the CAR cells and potentially induce adaptive T cell responses even in nutrient-poor environments. In embodiments, the disclosed methods and compositions take advantage of the phagocytic nature of macrophages to deposit slowly releasing F16BP metabolite formulation within macrophages or dendritic cells to provide energy to the CAR-macrophages. Activated CAR-macrophages can then infiltrate tumors, and once in the tumor, cancer cells actively prevent the activation of macrophages, by generating an immunosuppressive microenvironment. In embodiments, macrophages are activated in a time-dependent manner, after/during their chemotaxis to the tumor microenvironment, and thus have a higher probability of killing the cancer cells, without getting suppressed.

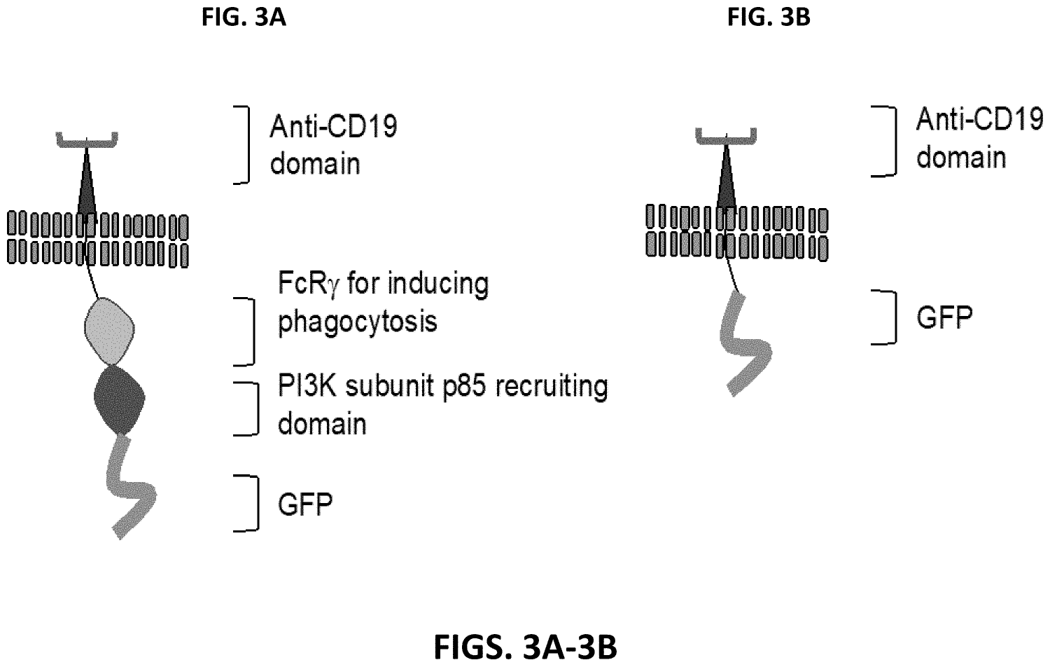

An exemplary CAR construct is provided in FIG. 3A. As illustrated in FIG. 3A, an exemplary CAR construct includes a CD19 single chain variable fragment receptor as the extracellular domain and intracellular domains of FcRgamma with p85 subunit of PI3K recruiting domain, and GFP reporter. FIG. 3B provides an empty construct with ScFv CD19 domain with GFP reporter.

In embodiments, CD19 CAR expressing plasmids are used for macrophage-based immunotherapy. Macrophages can exist in two forms, activated M1, and suppressive M2, and tumor microenvironment actively promotes M2 phenotype. Enhancing M1/M2 ratio leads to a better outcome. The disclosed metabolite-based particles can be used to maintain M1 phenotype in solid B cell lymphoma tumor environment.

In embodiments, CAR-macrophages are generated using plasmids with enhanced phagocytic ability based on the principals disclosed in Morrissey et. al. (Elife (2018). doi:10.7554/elife.36688), which is hereby incorporated by reference in its entirety. In some embodiments, the genes of interest from pHR backbone are sub-cloned into a vector construct, such as pCDNA3.1 backbone with geneticin (G418) and ampicillin selection to isolate cells expressing CAR construct and verified using sequencing data. Specifically, both CAR plasmids include an extracellular single chain fragment variable region of a receptor capable of binding CD19 on B cells. Moreover, intracellularly, FcRgamma domain was added to the Tandem construct capable of promoting phagocytosis. In addition to FcRgamma domain, p85, a subunit of PI3K protein, recruiting domain was also added to Tandem. PI3K recruits NADPH oxidase (for reactive oxygen release) on the membrane, and may also play a part in macrophage or dendritic cell spreading, chemotaxis, and phagocytosis.

The CARs as described herein can include an extracellular target-specific binding domain, a transmembrane domain, an intracellular signaling domain (such as a signaling domain FcRgamma), one or more co-stimulatory signaling domains derived from a co-stimulatory molecule, such as, but not limited to, intracellular p85-mediated PI3K recruiting domain and a glycolysis accelerating metabolite, such as, but not limited to, F6P, G6P, PVP, F16BP, or succinate.

The CAR can include a hinge or spacer region between the extracellular binding domain and the transmembrane domain, such as a CD8alpha hinge.

The binding domain or the extracellular domain of the CAR provides the CAR with the ability to bind to the target antigen of interest. A binding domain (e.g., a ligand-binding domain or antigen-binding domain) can be any protein, polypeptide, oligopeptide, or peptide that possesses the ability to specifically recognize and bind to a biological molecule (e.g., a cell surface receptor or tumor protein, or a component thereof), such as the antigen binding domain of an auto antigen described and/or detected using the methods described here. A binding domain includes any naturally occurring, synthetic, semi-synthetic, or recombinantly produced binding partner for a biological molecule of interest. For example, a binding domain may be antibody light chain and heavy chain variable regions, or the light and heavy chain variable regions can be joined together in a single chain and in either orientation (e.g., VL-VH or VH-VL). A variety of assays are known for identifying binding domains of the present disclosure that specifically bind with a particular target, including Western blot, ELISA, flow cytometry, or surface plasmon resonance analysis (e.g., using BIACORE analysis). The target may be an antigen of clinical interest against which it would be desirable to trigger an effector immune response that results in tumor killing.

Illustrative ligand-binding domains include antigen binding proteins, such as antigen binding fragments of an antibody, such as scFv, scTCR, extracellular domains of receptors, ligands for cell surface molecules/receptors, or receptor binding domains thereof, and tumor binding proteins. In certain embodiments, the antigen binding domains included in a CAR can be a variable region (Fv), a CDR, a Fab, an scFv, a VH, a VL, a domain antibody variant (dAb), a camelid antibody (VHH), a fibronectin 3 domain variant, an ankyrin repeat variant and other antigen-specific binding domain derived from other protein scaffolds.

In one embodiment, the binding domain of the CAR is a single chain antibody (scFv), and may be a murine, human or humanized scFv. Single chain antibodies may be cloned from the V region genes of a hybridoma specific for a desired target. A technique which can be used for cloning the variable region heavy chain (VH) and variable region light chain (VL) has been described, for example, in Orlandi et al., PNAS, 1989; 86: 3833-3837. Thus, in certain embodiments, a binding domain comprises an antibody-derived binding domain but can be a non-antibody derived binding domain. An antibody-derived binding domain can be a fragment of an antibody or a genetically engineered product of one or more fragments of the antibody, which fragment is involved in binding with the antigen.

In certain embodiments, the CARs can include a linker(s) between the various domains, added for appropriate spacing and conformation of the molecule. For example, in one embodiment, there may be a linker between the binding domain VH or VL which may be between 1-10 amino acids long. In other embodiments, the linker between any of the domains of the chimeric antigen receptor may be between 1-20 or 20 amino acids long. In this regard, the linker may be 1, 2, 3, 4, 5, 6, 7, 8, 9, 10, 11, 12, 13, 14, 15, 16, 17, 18, 19 or 20 amino acids long. In further embodiments, the linker may be 21, 22, 23, 24, 25, 26, 27, 28, 29 or 30 amino acids long.

In certain embodiments, linkers suitable for use in the CAR are flexible linkers. Suitable linkers can be readily selected and can be of any of a suitable of different lengths, such as from 1 amino acid (e.g., Gly) to 20 amino acids, from 2 amino acids to 15 amino acids, from 3 amino acids to 12 amino acids, including 4 amino acids to 10 amino acids, 5 amino acids to 9 amino acids, 6 amino acids to 8 amino acids, or 7 amino acids to 8 amino acids, and may be 1, 2, 3, 4, 5, 6, or 7 amino acids.

Exemplary flexible linkers include glycine polymers (G)n, glycine-serine polymers, where n is an integer of at least one, glycine-alanine polymers, alanine-serine polymers, and other flexible linkers known in the art. Glycine and glycine-serine polymers are relatively unstructured, and therefore may be able to serve as a neutral tether between domains of fusion proteins such as the CARs described herein. Glycine accesses significantly more phi-psi space than even alanine, and is much less restricted than residues with longer side chains (see Scheraga, Rev. Computational Chem. 11173-142 (1992)). The ordinarily skilled artisan will recognize that design of a CAR can include linkers that are all or partially flexible, such that the linker can include a flexible linker as well as one or more portions that confer less flexible structure to provide for a desired CAR structure.

The binding domain of the CAR can be followed by a “spacer,” or, “hinge,” which refers to the region that moves the antigen binding domain away from the effector cell surface to enable proper cell/cell contact, antigen binding and activation (Patel et al., Gene Therapy, 1999; 6: 412-419). The hinge region in a CAR is generally between the transmembrane (TM) and the binding domain. In certain embodiments, a hinge region is an immunoglobulin hinge region and may be a wild type immunoglobulin hinge region or an altered wild type immunoglobulin hinge region. Other exemplary hinge regions used in the CARs described herein include the hinge region derived from the extracellular regions of type 1 membrane proteins such as CD8alpha, CD4, CD28 and CD7, which may be wild-type hinge regions from these molecules or may be altered.

The “transmembrane” region or domain is the portion of the CAR that anchors the extracellular binding portion to the plasma membrane of the immune effector cell, and facilitates binding of the binding domain to the target antigen. The transmembrane domain may be a CD3zeta transmembrane domain, however other transmembrane domains that may be employed include those obtained from CD8alpha, CD4, CD28, CD45, CD9, CD16, CD22, CD33, CD64, CD80, CD86, CD134, CD137, and CD154.

The “intracellular signaling domain” or “signaling domain” refers to the part of the chimeric antigen receptor protein that participates in transducing the message of effective CAR binding to a target antigen into the interior of the immune effector cell to elicit effector cell function, e.g., activation, cytokine production, proliferation and cytotoxic activity, including the release of cytotoxic factors to the CAR-bound target cell, or other cellular responses elicited with antigen binding to the extracellular CAR domain. The term “effector function” refers to a specialized function of the cell. Thus, the terms “intracellular signaling domain” or “signaling domain,” used interchangeably herein, refer to the portion of a protein which transduces the effector function signal and that directs the cell to perform a specialized function. While usually the entire intracellular signaling domain can be employed, in many cases it is not necessary to use the entire domain. To the extent that a truncated portion of an intracellular signaling domain is used, such truncated portion may be used in place of the entire domain as long as it transduces the effector function signal. The term intracellular signaling domain is meant to include any truncated portion of the intracellular signaling domain sufficient to transducing effector function signal. The intracellular signaling domain is also known as the “signal transduction domain,” and is typically derived from portions of the human CD3 or FcRy chains.

In embodiments, a disclosed CAR includes one or more cytoplasmic signaling sequences that act in a costimulatory manner may contain signaling motifs which are known as immunoreceptor tyrosine-based activation motif or ITAMs.

Examples of ITAM containing primary cytoplasmic signaling sequences that are of particular use in the invention include those derived from TCRzeta, FcRgamma, FcRbeta, CD3gamma, CD3delta, CD3epsilon, CD5, CD22, CD79a, CD79b and CD66d. In one particular embodiment, the intracellular signaling domain of FcRgamma.

As used herein, the term, “costimulatory signaling domain,” or “costimulatory domain”, refers to the portion of the CAR comprising the intracellular domain of a costimulatory molecule. Costimulatory molecules are cell surface molecules other than antigen receptors or Fc receptors that provide a second signal. Examples of such co-stimulatory molecules include, but are not limited to, CD27, CD28, 4-1BB (CD137), OX40 (CD134), CD30, CD40, PD-1, ICOS (CD278), LFA-1, CD2, CD7, LIGHT, NKD2C, B7-H2 and a ligand that specifically binds CD83. Accordingly, while the present disclosure provides exemplary costimulatory domain, p85-mediated PI3K recruiting domain, other costimulatory domains are contemplated for use with the CARs described herein. The inclusion of one or more co-stimulatory signaling domains may enhance the efficacy of the macrophages expressing CAR receptors. The intracellular signaling and costimulatory signaling domains may be linked in any order in tandem to the carboxyl terminus of the transmembrane domain.

Some disclosed scFv-based CARs are engineered to contain a signaling domain from CD3 or FcRgamma. Other CARs contain a binding domain, a hinge, a transmembrane and the signaling domain derived from FcRgamma or CD3 together with one or more costimulatory signaling domains.

In certain embodiments, the polynucleotide encoding the CAR described herein is inserted into a vector. The vector is a vehicle into which a polynucleotide encoding a protein may be covalently inserted so as to bring about the expression of that protein and/or the cloning of the polynucleotide. Such vectors may also be referred to as “expression vectors”. The isolated polynucleotide may be inserted into a vector using any suitable methods known in the art, for example, without limitation, the vector may be digested using appropriate restriction enzymes and then may be ligated with the isolated polynucleotide having matching restriction ends. Expression vectors have the ability to incorporate and express heterologous or modified nucleic acid sequences coding for at least part of a gene product capable of being transcribed in a cell. In most cases, RNA molecules are then translated into a protein. Expression vectors can contain a variety of control sequences, which refer to nucleic acid sequences necessary for the transcription and possibly translation of an operatively linked coding sequence in a particular host organism. In addition to control sequences that govern transcription and translation, vectors and expression vectors may contain nucleic acid sequences that serve other functions as well and are discussed infra. An expression vector may comprise additional elements, for example, the expression vector may have two replication systems, thus allowing it to be maintained in two organisms, for example in human cells for expression and in a prokaryotic host for cloning and amplification.

The expression vector may have the necessary 5′ upstream and 3′ downstream regulatory elements such as promoter sequences such as CMV, PGK and EF1alpha. promoters, ribosome recognition and binding TATA box, and 3′ UTR AAUAAA transcription termination sequence for the efficient gene transcription and translation in its respective host cell. Other suitable promoters include the constitutive promoter of simian virus 40 (SV40) early promoter, mouse mammary tumor virus (MMTV), HIV LTR promoter, MoMuLV promoter, avian leukemia virus promoter, EBV immediate early promoter, and rous sarcoma virus promoter. Human gene promoters may also be used, including, but not limited to the actin promoter, the myosin promoter, the hemoglobin promoter, and the creatine kinase promoter. In certain embodiments inducible promoters are also contemplated as part of the vectors expressing chimeric antigen receptor. This provides a molecular switch capable of turning on expression of the polynucleotide sequence of interest or turning off expression. Examples of inducible promoters include, but are not limited to a metallothionine promoter, a glucocorticoid promoter, a progesterone promoter, or a tetracycline promoter.

The expression vector may have additional sequence such as GFP, 6×-histidine, c-Myc, and FLAG tags which are incorporated into the expressed CARs. Thus, the expression vector may be engineered to contain 5′ and 3′ untranslated regulatory sequences that sometimes can function as enhancer sequences, promoter regions and/or terminator sequences that can facilitate or enhance efficient transcription of the nucleic acid(s) of interest carried on the expression vector. An expression vector may also be engineered for replication and/or expression functionality (e.g., transcription and translation) in a particular cell type, cell location, or tissue type. Expression vectors may include a selectable marker for maintenance of the vector in the host or recipient cell.

In various embodiments, the vectors are plasmid, autonomously replicating sequences, and transposable elements. Additional exemplary vectors include, without limitation, plasmids, phagemids, cosmids, artificial chromosomes such as yeast artificial chromosome (YAC), bacterial artificial chromosome (BAC), or P1-derived artificial chromosome (PAC), bacteriophages such as lambda phage or M13 phage, and animal viruses. Examples of categories of animal viruses useful as vectors include, without limitation, retrovirus (including lentivirus), adenovirus, adenoassociated virus, herpesvirus (e.g., herpes simplex virus), poxvirus, baculovirus, papillomavirus, and papovavirus (e.g., SV40). Examples of expression vectors are Lenti-X™ Bicistronic Expression System (Neo) vectors (Clontrch), pClneo vectors (Promega) for expression in mammalian cells; pLenti4/V5-DEST™, pLenti6/V5-DEST™, and pLenti6.2N5-GW/lacZ (Invitrogen) for lentivirus-mediated gene transfer and expression in mammalian cells. The coding sequences of the CARs disclosed herein can be ligated into such expression vectors for the expression of the chimeric protein in mammalian cells.

In certain embodiments, the nucleic acids encoding the CAR are provided in a viral vector. A viral vector can be that derived from retrovirus, lentivirus, or foamy virus. As used herein, the term, “viral vector,” refers to a nucleic acid vector construct that includes at least one element of viral origin and has the capacity to be packaged into a viral vector particle. The viral vector can contain the coding sequence for the various chimeric proteins described herein in place of nonessential viral genes. The vector and/or particle can be utilized for the purpose of transferring DNA, RNA or other nucleic acids into cells either in vitro or in vivo. Numerous forms of viral vectors are known in the art.

In certain embodiments, the CAR-phagocyte can be utilized to deliver the virus to the tumor. For example, CAR-phagocytes can be loaded with oncolytic virus and injected for enhanced viral replication and delivery to the tumor. One such example of oncolytic virus can be MYVX virus (Myxoma virus).

In certain embodiments, the viral vector containing the coding sequence for a CAR described herein is a retroviral vector or a lentiviral vector. The term “retroviral vector” refers to a vector containing structural and functional genetic elements that are primarily derived from a retrovirus. The term “lentiviral vector” refers to a vector containing structural and functional genetic elements outside the LTRs that are primarily derived from a lentivirus.

The retroviral vectors for use herein can be derived from any known retrovirus (e.g., type c retroviruses, such as Moloney murine sarcoma virus (MoMSV), Harvey murine sarcoma virus (HaMuSV), murine mammary tumor virus (MuMTV), gibbon ape leukemia virus (GaLV), feline leukemia virus (FLV), spumavirus, Friend, Murine Stem Cell Virus (MSCV) and Rous Sarcoma Virus (RSV)). Retroviruses” of the invention also include human T cell leukemia viruses, HTLV-1 and HTLV-2, and the lentiviral family of retroviruses, such as Human Immunodeficiency Viruses, HIV-1, HIV-2, simian immunodeficiency virus (SIV), feline immunodeficiency virus (FIV), equine immunodeficiency virus (EIV), and other classes of retroviruses.

A lentiviral vector for use herein refers to a vector derived from a lentivirus, a group (or genus) of retroviruses that give rise to slowly developing disease. Viruses included within this group include HIV (human immunodeficiency virus; including HIV type 1, and HIV type 2); visna-maedi; a caprine arthritis-encephalitis virus; equine infectious anemia virus; feline immunodeficiency virus (FIV); bovine immune deficiency virus (BIV); and simian immunodeficiency virus (SIV). Preparation of the recombinant lentivirus can be achieved using the methods according to Dull et al. and Zufferey et al. (Dull et al., J. Virol., 1998; 72: 8463-8471 and Zufferey et al., J. Virol. 1998; 72:9873-9880).

Retroviral vectors (i.e., both lentiviral and non-lentiviral) for use in the present invention can be formed using standard cloning techniques by combining the desired DNA sequences in the order and orientation described herein (Current Protocols in Molecular Biology, Ausubel, F. M. et al., (eds.) Greene Publishing Associates, (1989), Sections 9.10-9.14 and other standard laboratory manuals; Eglitis, et al., (1985) Science 230:1395-1398; Danos and Mulligan (1988) Proc. Natl. Acad. Sci. USA 85:6460-6464; Wilson et al., (1988) Proc. Natl. Acad. Sci. USA 85:3014-3018; Armentano et al., (1990) Proc. Natl. Acad. Sci. USA 87:6141-6145; Huber et al., (1991) Proc. Natl. Acad. Sci. USA 88:8039-8043; Ferry et al., (1991) Proc. Natl. Acad. Sci. USA 88:8377-8381; Chowdhury et al., (1991) Science 254:1802-1805; van Beusechem et al. (1992) Proc. Natl. Acad. Sci. USA 89:7640-7644; Kay et al., (1992) Human Gene Therapy 3:641-647; Dai et al., (1992) Proc. Natl. Acad. Sci. USA 89:10892-10895; Hwu et al., (1993) J. Immunol 150:4104-4115; U.S. Pat. Nos. 4,868,116; 4,980,286; PCT Application WO 89/07136; PCT Application WO 89/02468; PCT Application WO 89/05345; and PCT Application WO 92/07573).

Suitable sources for obtaining retroviral (i.e., both lentiviral and non-lentiviral) sequences for use in forming the vectors include, for example, genomic RNA and cDNAs available from commercially available sources, including the Type Culture Collection (ATCC), Rockville, Md. The sequences also can be synthesized chemically.

For expression of a CAR, the vector may be introduced into a host cell to allow expression of the polypeptide within the host cell. The expression vectors may contain a variety of elements for controlling expression, including without limitation, promoter sequences, transcription initiation sequences, enhancer sequences, selectable markers, and signal sequences. These elements may be selected as appropriate by a person of ordinary skill in the art, as described above. For example, the promoter sequences may be selected to promote the transcription of the polynucleotide in the vector. Suitable promoter sequences include, without limitation, T7 promoter, T3 promoter, SP6 promoter, beta-actin promoter, EF1a promoter, CMV promoter, and SV40 promoter. Enhancer sequences may be selected to enhance the transcription of the polynucleotide. Selectable markers may be selected to allow selection of the host cells inserted with the vector from those not, for example, the selectable markers may be genes that confer antibiotic resistance. Signal sequences may be selected to allow the expressed polypeptide to be transported outside of the host cell.

For cloning of the polynucleotide, the vector may be introduced into a host cell (an isolated host cell) to allow replication of the vector itself and thereby amplify the copies of the polynucleotide contained therein. The cloning vectors may contain sequence components generally include, without limitation, an origin of replication, promoter sequences, transcription initiation sequences, enhancer sequences, and selectable markers. These elements may be selected as appropriate by a person of ordinary skill in the art. For example, the origin of replication may be selected to promote autonomous replication of the vector in the host cell.

In certain embodiments, the present disclosure provides isolated host cells containing the vectors provided herein. The host cells containing the vector may be useful in expression or cloning of the polynucleotide contained in the vector. Suitable host cells can include, without limitation, prokaryotic cells, fungal cells, yeast cells, or higher eukaryotic cells such as mammalian cells. Suitable prokaryotic cells for this purpose include, without limitation, eubacteria, such as Gram-negative or Gram-positive organisms, for example, Enterobactehaceae such as Escherichia, e.g., E. coli, Enterobacter, Envinia, Klebsiella, Proteus, Salmonella, e.g., Salmonella typhimurium, Serratia, e.g., Serratia marcescans, and Shigella, as well as Bacilli such as B. subtilis and B. licheniformis, Pseudomonas such as P. aeruginosa, and Streptomyces.

The CARs are introduced into a host cell using transfection and/or transduction techniques known in the art. As used herein, the terms, “transfection,” and, “transduction,” refer to the processes by which an exogenous nucleic acid sequence is introduced into a host cell. The nucleic acid may be integrated into the host cell DNA or may be maintained extrachromosomally. The nucleic acid may be maintained transiently or may be a stable introduction. Transfection may be accomplished by a variety of means known in the art including but not limited to calcium phosphate-DNA co-precipitation, DEAE-dextran-mediated transfection, polybrene-mediated transfection, electroporation, microinjection, liposome fusion, lipofection, protoplast fusion, retroviral infection, and biolistics. Transduction refers to the delivery of a gene(s) using a viral or retroviral vector by means of viral infection rather than by transfection. In certain embodiments, retroviral vectors are transduced by packaging the vectors into virions prior to contact with a cell. For example, a nucleic acid encoding a CAR carried by a retroviral vector can be transduced into a cell through infection and pro virus integration.

As used herein, the term “genetically engineered” or “genetically modified” refers to the addition of extra genetic material in the form of DNA or RNA into the total genetic material in a cell. The terms, “genetically modified cells,” “modified cells,” and, “redirected cells,” are used interchangeably.

In particular, the CAR is introduced and expressed in immune effector cells so as to redirect their specificity to a target antigen of interest, e.g., a tumor cell.

Methods for making the immune effector cells which express the CAR are provided. In one embodiment, the method comprises transfecting or transducing immune effector cells isolated from a subject, such as a subject having a solid or diffuse tumor, such that the immune effector cells express one or more CAR as described herein. In certain embodiments, the immune effector cells are isolated from an individual and genetically modified without further manipulation in vitro. Such cells can then be directly re-administered into the individual. In further embodiments, the immune effector cells are first activated and stimulated to proliferate in vitro prior to being genetically modified to express a CAR. In this regard, the immune effector cells may be cultured before or after being genetically modified (i.e., transduced or transfected to express a CAR as described herein).

Prior to in vitro manipulation or genetic modification of the immune effector cells described herein, the source of cells may be obtained from a subject or a cell line can be utilized. In particular, the immune effector cells for use with the CARs as described herein comprise macrophage or dendritic cells (DCs). Macrophage cells or DCs can be obtained from a number of source, processed (such as washed) and isolated. The immune effector cells, such as macrophage cells, can be genetically modified following isolation using known methods, or the immune effector cells can be activated and expanded (or differentiated in the case of progenitors) in vitro prior to being genetically modified. In another embodiment, the immune effector cells, are genetically modified with the chimeric antigen receptors described herein (e.g., transduced with a viral vector comprising a nucleic acid encoding a CAR) and then are activated and expanded in vitro.

The invention provides a population of modified immune effector cells for the treatment of a patient having a solid or diffuse tumor, such as lymphoma and/or leukemia tumors, breast cancer, melanoma, or sarcomas. In some examples, the cancer is Acanthoma, Acinic cell carcinoma, Acoustic neuroma, Acral lentiginous melanoma, Acrospiroma, Acute eosinophilic leukemia, Acute lymphoblastic leukemia, Acute megakaryoblastic leukemia, Acute monocytic leukemia, Acute myeloblastic leukemia with maturation, Acute myeloid dendritic cell leukemia, Acute myeloid leukemia, Acute promyelocytic leukemia, Adamantinoma, Adenocarcinoma, Adenoid cystic carcinoma, Adenoma, Adenomatoid odontogenic tumor, Adrenocortical carcinoma, Adult T-cell leukemia, Aggressive NK-cell leukemia, AIDS-Related Cancers, AIDS-related lymphoma, Alveolar soft part sarcoma, Ameloblastic fibroma, Anal cancer, Anaplastic large cell lymphoma, Anaplastic thyroid cancer, Angioimmunoblastic T-cell lymphoma, Angiomyolipoma, Angiosarcoma, Appendix cancer, Astrocytoma, Atypical teratoid rhabdoid tumor, Basal cell carcinoma, Basal-like carcinoma, B-cell leukemia, B-cell lymphoma, Bellini duct carcinoma, Biliary tract cancer, Bladder cancer, Blastoma, Bone Cancer, Bone tumor, Brain Stem Glioma, Brain Tumor, Breast Cancer, Brenner tumor, Bronchial Tumor, Bronchioloalveolar carcinoma, Brown tumor, Burkitt's lymphoma, Cancer of Unknown Primary Site, Carcinoid Tumor, Carcinoma, Carcinoma in situ, Carcinoma of the penis, Carcinoma of Unknown Primary Site, Carcinosarcoma, Castleman's Disease, Central Nervous System Embryonal Tumor, Cerebellar Astrocytoma, Cerebral Astrocytoma, Cervical Cancer, Cholangiocarcinoma, Chondroma, Chondrosarcoma, Chordoma, Choriocarcinoma, Choroid plexus papilloma, Chronic Lymphocytic Leukemia, Chronic monocytic leukemia, Chronic myelogenous leukemia, Chronic Myeloproliferative Disorder, Chronic neutrophilic leukemia, Clear-cell tumor, Colon Cancer, Colorectal cancer, Craniopharyngioma, Cutaneous T-cell lymphoma, Degos disease, Dermatofibrosarcoma protuberans, Dermoid cyst, Desmoplastic small round cell tumor, Diffuse large B cell lymphoma, Dysembryoplastic neuroepithelial tumor, Embryonal carcinoma, Endodermal sinus tumor, Endometrial cancer, Endometrial Uterine Cancer, Endometrioid tumor, Enteropathy-associated T-cell lymphoma, Ependymoblastoma, Ependymoma, Epithelioid sarcoma, Erythroleukemia, Esophageal cancer, Esthesioneuroblastoma, Ewing Family of Tumor, Ewing Family Sarcoma, Ewing's sarcoma, Extracranial Germ Cell Tumor, Extragonadal Germ Cell Tumor, Extrahepatic Bile Duct Cancer, Extramammary Paget's disease, Fallopian tube cancer, Fetus in fetu, Fibroma, Fibrosarcoma, Follicular lymphoma, Follicular thyroid cancer, Gallbladder Cancer, Ganglioglioma, Ganglioneuroma, Gastric Cancer, Gastric lymphoma, Gastrointestinal cancer, Gastrointestinal Carcinoid Tumor, Gastrointestinal Stromal Tumor, Germ cell tumor, Germinoma, Gestational choriocarcinoma, Gestational Trophoblastic Tumor, Giant cell tumor of bone, Glioblastoma multiforme, Glioma, Gliomatosis cerebri, Glomus tumor, Glucagonoma, Gonadoblastoma, Granulosa cell tumor, Hairy Cell Leukemia, Hairy cell leukemia, Head and Neck Cancer, Head and neck cancer, Heart cancer, Hemangioblastoma, Hemangiopericytoma, Hemangiosarcoma, Hematological malignancy, Hepatocellular carcinoma, Hepatosplenic T-cell lymphoma, Hereditary breast-ovarian cancer syndrome, Hodgkin Lymphoma, Hodgkin's lymphoma, Hypopharyngeal Cancer, Hypothalamic Glioma, Inflammatory breast cancer, Intraocular Melanoma, Islet cell carcinoma, Islet Cell Tumor, Juvenile myelomonocytic leukemia, Kaposi Sarcoma, Kaposi's sarcoma, Kidney Cancer, Klatskin tumor, Krukenberg tumor, Laryngeal Cancer, Laryngeal cancer, Lentigo maligna melanoma, Leukemia, Lip and Oral Cavity Cancer, Liposarcoma, Lung cancer, Luteoma, Lymphangioma, Lymphangiosarcoma, Lymphoepithelioma, Lymphoid leukemia, Lymphoma, Macroglobulinemia, Malignant Fibrous Histiocytoma, Malignant fibrous histiocytoma, Malignant Fibrous Histiocytoma of Bone, Malignant Glioma, Malignant Mesothelioma, Malignant peripheral nerve sheath tumor, Malignant rhabdoid tumor, Malignant triton tumor, MALT lymphoma, Mantle cell lymphoma, Mast cell leukemia, Mediastinal germ cell tumor, Mediastinal tumor, Medullary thyroid cancer, Medulloblastoma, Medulloepithelioma, Melanoma, Meningioma, Merkel Cell Carcinoma, Mesothelioma, Metastatic Squamous Neck Cancer with Occult Primary, Metastatic urothelial carcinoma, Mixed Mullerian tumor, Monocytic leukemia, Mouth Cancer, Mucinous tumor, Multiple Endocrine Neoplasia Syndrome, Multiple myeloma, Mycosis Fungoides, Myelodysplastic Disease, Myelodysplastic Syndromes, Myeloid leukemia, Myeloid sarcoma, Myeloproliferative Disease, Myxoma, Nasal Cavity Cancer, Nasopharyngeal Cancer, Nasopharyngeal carcinoma, Neoplasm, Neurinoma, Neuroblastoma, Neurofibroma, Neuroma, Nodular melanoma, Non-Hodgkin lymphoma, Nonmelanoma Skin Cancer, Non-Small Cell Lung Cancer, Ocular oncology, Oligoastrocytoma, Oligodendroglioma, Oncocytoma, Optic nerve sheath meningioma, Oral cancer, Oropharyngeal Cancer, Osteosarcoma, Ovarian cancer, Ovarian Epithelial Cancer, Ovarian Germ Cell Tumor, Ovarian Low Malignant Potential Tumor, Paget's disease of the breast, Pancoast tumor, Pancreatic cancer, Papillary thyroid cancer, Papillomatosis, Paraganglioma, Paranasal Sinus Cancer, Parathyroid Cancer, Penile Cancer, Perivascular epithelioid cell tumor, Pharyngeal Cancer, Pheochromocytoma, Pineal Parenchymal Tumor of Intermediate Differentiation, Pineoblastoma, Pituicytoma, Pituitary adenoma, Pituitary tumor, Plasma Cell Neoplasm, Pleuropulmonary blastoma, Polyembryoma, Precursor T-lymphoblastic lymphoma, Primary central nervous system lymphoma, Primary effusion lymphoma, Primary Hepatocellular Cancer, Primary Liver Cancer, Primary peritoneal cancer, Primitive neuroectodermal tumor, Prostate cancer, Pseudomyxoma peritonei, Rectal Cancer, Renal cell carcinoma, Respiratory Tract Carcinoma Involving the NUT Gene on Chromosome 15, Retinoblastoma, Rhabdomyoma, Rhabdomyosarcoma, Richter's transformation, Sacrococcygeal teratoma, Salivary Gland Cancer, Sarcoma, Schwannomatosis, Sebaceous gland carcinoma, Secondary neoplasm, Seminoma, Serous tumor, Sertoli-Leydig cell tumor, Sex cord-stromal tumor, Sezary Syndrome, Signet ring cell carcinoma, Skin Cancer, Small blue round cell tumor, Small cell carcinoma, Small Cell Lung Cancer, Small cell lymphoma, Small intestine cancer, Soft tissue sarcoma, Somatostatinoma, Soot wart, Spinal Cord Tumor, Spinal tumor, Splenic marginal zone lymphoma, Squamous cell carcinoma, Stomach cancer, Superficial spreading melanoma, Supratentorial Primitive Neuroectodermal Tumor, Surface epithelial-stromal tumor, Synovial sarcoma, T-cell acute lymphoblastic leukemia, T-cell large granular lymphocyte leukemia, T-cell leukemia, T-cell lymphoma, T-cell prolymphocytic leukemia, Teratoma, Terminal lymphatic cancer, Testicular cancer, Thecoma, Throat Cancer, Thymic Carcinoma, Thymoma, Thyroid cancer, Transitional Cell Cancer of Renal Pelvis and Ureter, Transitional cell carcinoma, Urachal cancer, Urethral cancer, Urogenital neoplasm, Uterine sarcoma, Uveal melanoma, Vaginal Cancer, Verner Morrison syndrome, Verrucous carcinoma, Visual Pathway Glioma, Vulvar Cancer, Waldenstrom's macroglobulinemia, Warthin's tumor, or Wilms' tumor.

CAR-expressing immune effector cells prepared as described herein can be utilized in methods and compositions for adoptive immunotherapy in accordance with known techniques, or variations thereof that will be apparent to those skilled in the art based on the instant disclosure. For example, in some embodiments, treatments and methods of the present disclosure can include adoptive cell therapy (ACT). In some embodiments, ACT can be performed with tumor-infiltrating lymphocytes (TIL) or gene-modified T cells expressing novel T cell receptors (TCR) or chimeric antigen receptors (CAR). ACT is used to modify the immune system to recognize tumor cells and thus carry out an anti-tumor effector function. In some embodiments, ACT can include the use of the glycolysis accelerating metabolites (e.g., microparticles) of the present disclosure, with or without the use of engineered immune cells expressing various TCRs or CARs. In some embodiments, ACT can include the use of the glycolysis accelerating metabolites (e.g., microparticles) of the present disclosure without the use of engineered immune cells expressing various TCRs or CARs for the treatment of ovarian cancer, melanoma, and lymphoma.

The CAR expressing immune effector cell populations of the present invention may be administered either alone, or as a pharmaceutical composition in combination with diluents and/or with other components such as IL-2 or other cytokines or cell populations. Briefly, pharmaceutical compositions of the present invention may comprise a CAR-expressing immune effector cell population as described herein, in combination with one or more pharmaceutically or physiologically acceptable carriers, diluents or excipients. Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. Compositions of the present invention are preferably formulated for intravenous administration.

The anti-tumor immune response induced in a subject by administering CAR expressing macrophages described herein using the methods described herein. A variety of techniques may be used for analyzing the type of immune responses induced by the compositions of the present invention, which are well described in the art; e.g., Current Protocols in Immunology, Edited by: John E. Coligan, Ada M. Kruisbeek, David H. Margulies, Ethan M. Shevach, Warren Strober (2001) John Wiley & Sons, NY, N.Y.

Thus, provided are methods of treating an individual diagnosed with or suspected of having, or at risk of developing a solid tumor, such as lymphoma and/or leukemia tumors as described herein.

In one embodiment, the invention provides a method of treating a subject diagnosed with lymphoma and/or leukemia tumor comprising removing immune effector cells from a subject diagnosed with lymphoma and/or leukemia, genetically modifying said immune effector cells with a vector comprising a nucleic acid encoding a chimeric antigen receptor of the instant invention, thereby producing a population of modified immune effector cells, and administering the population of modified immune effector cells to the same subject. In one embodiment, the immune effector cells comprise macrophages.

The methods for administering the cell compositions described herein includes any method which is effective to result in reintroduction of ex vivo genetically modified immune effector cells that either directly express a CAR of the invention in the subject or on reintroduction of the genetically modified progenitors of immune effector cells that on introduction into a subject differentiate into mature immune effector cells that express the CAR. One method comprises transducing macrophages ex vivo with a nucleic acid construct in accordance with the invention and returning the transduced cells into the subject.

Disclosed are methods of preparing immune cells for immunotherapy comprising introducing, ex vivo, into such immune cells the polynucleotides or vectors encoding one of the chimeric antigen receptors described herein.

The present invention also encompasses immune cells comprising a polynucleotide or lentiviral vector encoding one of the chimeric antigen receptors discussed herein. In some embodiments, these immune cells are used for immunotherapy (e.g., treatment of cancer).

### Engineered Immune Cells

Immune cells comprising a chimeric antigen receptor of the invention (or engineered immune cells) are another aspect of the present invention. In some cases, the immune cell is an immune effector cell, such as a T lymphocyte or T cell. In some cases, the immune cell is a macrophage or neutrophil or dendritic cell. In some cases, the immune cell is a natural killer cell, including but not limited to, an NK-92 cell.

### Activation and Expansion of Engineered Immune Cells

Whether prior to or after genetic modification of the engineered cells (e.g., macrophages or neutrophils, dendritic cells, T cells and/or NK cells), even if the genetically modified immune cells of the present invention are activated and proliferate independently of antigen binding mechanisms, the immune cells, can be further activated and expanded in vitro or in vivo.

### Therapeutic Applications

The present invention includes compositions comprising an engineered cell expressing a chimeric antigen receptor of the invention and a pharmaceutically acceptable vehicle. In some cases, the engineered cells form a medicament, particularly for immunotherapy. In some cases, the engineered cells are used for the treatment of cancer (e.g., lymphoma and/or leukemia). In some cases, the engineered cells are used in the manufacture of a medicament for immunotherapy and/or the treatment of lymphoma and/or leukemia tumors.

The present invention includes methods comprising administering to a subject in need thereof a therapeutic composition comprising an engineered cell expressing a chimeric antigen receptor as discussed herein. The therapeutic composition can comprise a cell expressing any chimeric antigen receptor as disclosed herein and a pharmaceutically acceptable carrier, diluent or vehicle. As used herein, the expression “a subject in need thereof” means a human or non-human animal that exhibits one or more symptoms or indicia of cancer (e.g., a subject expressing a tumor or suffering from any of the cancers mentioned herein), or who otherwise would benefit from an inhibition or reduction or a depletion of lymphoma and/or leukemia tumor cells.

The engineered cells of the present invention are useful, inter alia, for treating any disease or disorder in which stimulation, activation and/or targeting of an immune response would be beneficial.

The present invention also includes methods for treating residual cancer in a subject. As used herein, the term “residual cancer” means the existence or persistence of one or more cancerous cells in a subject following treatment with an anti-cancer therapy. The present invention also includes methods for treating metastatic cancer in a subject.

According to certain aspects, the present invention provides methods for treating a tumor, such as lymphoma and/or leukemia tumors, comprising administering a population of engineered cells described elsewhere herein to a subject after the subject has been determined to have lymphoma and/or leukemia tumor. For example, the present invention includes methods for treating comprising administering engineered immune cells to a patient 1 day, 2 days, 3 days, 4 days, 5 days, 6 days, 1 week, 2 weeks, 3 weeks or 4 weeks, 2 months, 4 months, 6 months, 8 months, 1 year, or more after the subject has received other immunotherapy or chemotherapy.

Cells that can be used with the disclosed methods are described herein. The treatments can be used to treat patients diagnosed with a tumor, lymphoma and/or leukemia tumors. The administration of the cells or population of cells according to the present invention may be carried out in any convenient manner, including by aerosol inhalation, injection, ingestion, transfusion, implantation or transplantation. The compositions described herein may be administered to a patient subcutaneously, intradermally, intratumorally, intranodally, intramedullary, intramuscularly, by intravenous or intralymphatic injection, or intraperitoneally. In one embodiment, the cell compositions of the present invention are preferably administered by intravenous injection.

The administration of the cells or population of cells can consist of the administration of 10⁴-10⁹ cells per kg body weight, preferably 10⁵ to 10⁶ cells/kg body weight including all integer values of cell numbers within those ranges. The cells or population of cells can be administered in one or more doses. In some embodiments, the effective amount of cells is administered as a single dose. In some embodiments, the effective amount of cells is administered as more than one dose over a period time. Timing of administration is within the judgment of managing physician and depends on the clinical condition of the patient. The cells or population of cells may be obtained from any source, such as a blood bank or a donor. While individual needs vary, determination of ranges of effective amounts of a given cell type for a particular disease or condition are within the skill of the art. An effective amount means an amount which provides a therapeutic or prophylactic benefit. The dosage administered will be dependent upon the age, health and weight of the recipient, kind of concurrent treatment, if any, frequency of treatment and the nature of the effect desired.

In one embodiment, the effective amount of cells or composition comprising those cells is administered parenterally. This administration can be an intravenous administration. In some cases, administration can be directly done by injection within a tumor.

In certain embodiments of the present invention, cells are administered to a patient in conjunction with (e.g., before, simultaneously or following) any number of relevant treatment modalities.

In some embodiments, the compositions described herein can be used for “off the shelf” immunotherapy. For example, CAR T cells and/or CAR NK cells can be engineered as provided herein and administered with a glycolysis accelerating metabolite as part of “off the shelf” immunotherapy. As would be recognized by one of ordinary skill in the art based on the present disclosure, this therapy involves engineering immune cells to avoid the deleterious allogenic immune responses that lead to toxicity and rejection (i.e., GvHD).

### Administration Regimens

According to certain embodiments of the present disclosure, multiple doses of the engineered cells may be administered to a subject over a defined time course. The methods according to this aspect of the disclosure comprise sequentially administering to a subject multiple doses of the cells. As used herein, “sequentially administering” means that each dose is administered to the subject at a different point in time, e.g., on different days separated by a predetermined interval (e.g., hours, days, weeks or months). The present disclosure includes methods which comprise sequentially administering to the patient a single initial dose, followed by one or more secondary doses, and optionally followed by one or more tertiary doses.

The terms “initial dose,” “secondary doses,” and “tertiary doses,” refer to the temporal sequence of administration of the engineered cells of the present disclosure. Thus, the “initial dose” is the dose which is administered at the beginning of the treatment regimen (also referred to as the “baseline dose”); the “secondary doses” are the doses which are administered after the initial dose; and the “tertiary doses” are the doses which are administered after the secondary doses. The initial, secondary, and tertiary doses may all contain the same amount of engineered cells, but generally may differ from one another in terms of frequency of administration. In certain embodiments, however, the amount of engineered cells contained in the initial, secondary and/or tertiary doses varies from one another (e.g., adjusted up or down as appropriate) during the course of treatment. In certain embodiments, two or more (e.g., 2, 3, 4, or 5) doses are administered at the beginning of the treatment regimen as “loading doses” followed by subsequent doses that are administered on a less frequent basis (e.g., “maintenance doses”).

In one exemplary embodiment of the present disclosure, each secondary and/or tertiary dose is administered 1 to 26 (e.g., 1, 1½, 2, 2½, 3, 3½, 4, 4½, 5, 5½, 6, 6½, 7, 7½, 8, 8½, 9, 9½, 10, 10½, 11, 11½, 12, 12½, 13, 13½, 14, 14½, 15, 15½, 16, 16½, 17, 17½, 18, 18½, 19, 19½, 20, 20½, 21, 21½, 22, 22½, 23, 23½, 24, 24½, 25, 25½, 26, 26½, or more) weeks after the immediately preceding dose. The phrase “the immediately preceding dose,” as used herein, means, in a sequence of multiple administrations, the dose which is administered to a patient prior to the administration of the very next dose in the sequence with no intervening doses.

The methods according to this aspect of the present disclosure may comprise administering to a patient any number of secondary and/or tertiary doses. For example, in certain embodiments, only a single secondary dose is administered to the patient. In other embodiments, two or more (e.g., 2, 3, 4, 5, 6, 7, 8, or more) secondary doses are administered to the patient. Likewise, in certain embodiments, only a single tertiary dose is administered to the patient. In other embodiments, two or more (e.g., 2, 3, 4, 5, 6, 7, 8, or more) tertiary doses are administered to the patient.

In embodiments involving multiple secondary doses, each secondary dose may be administered at the same frequency as the other secondary doses. For example, each secondary dose may be administered to the patient 1 to 2 weeks after the immediately preceding dose. Similarly, in embodiments involving multiple tertiary doses, each tertiary dose may be administered at the same frequency as the other tertiary doses. For example, each tertiary dose may be administered to the patient 2 to 4 weeks after the immediately preceding dose. Alternatively, the frequency at which the secondary and/or tertiary doses are administered to a patient can vary over the course of the treatment regimen. The frequency of administration may also be adjusted during the course of treatment by a physician depending on the needs of the individual patient following clinical examination.

The disclosure is further illustrated by the following non-limiting Examples.

### EXAMPLES

### Example 1

### F16BP Particle-Loaded CAR-Macs Modulate Adaptive Immune Responses In Vitro

This Example illustrates that metabolically-fit CAR-macs will phagocytose and kill Ramos B lymphoma cells in vitro and generate adaptive immune responses.

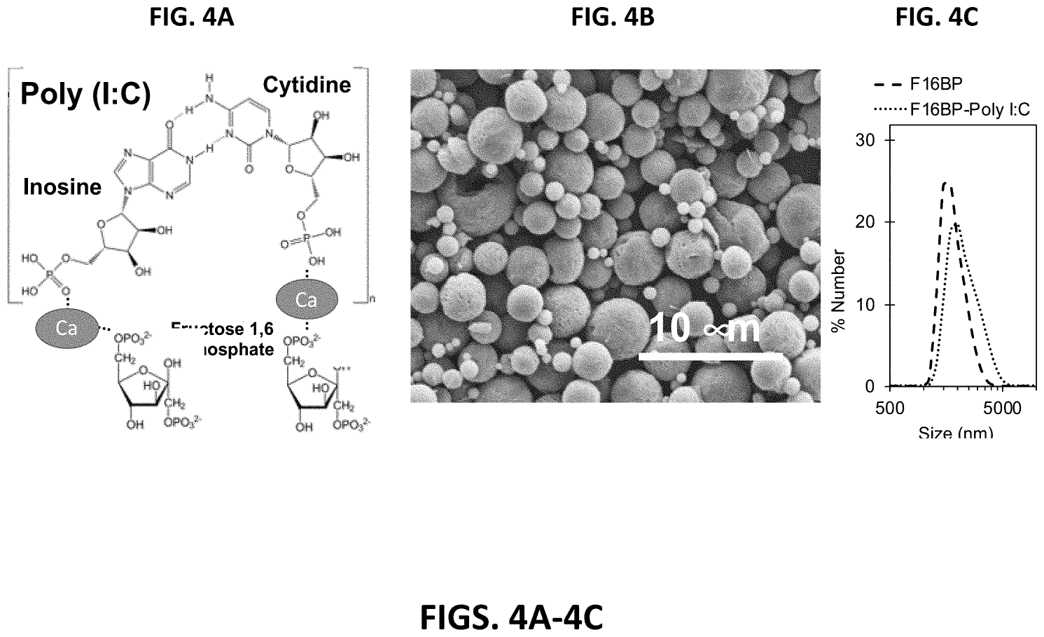

F16BP can be formulated in particles incorporating poly(I:C) adjuvant. To facilitate phagocytosis, and ensure that the F16BP metabolite is delivered intracellularly, formulations were generated in a particle format. Novel particles were synthesized using calcium-phosphate ionic bond chemistry. Schematic of the polymer structure formed between the phosphate groups of F16BP (a key metabolite in glycolysis) and calcium (FIG. 4A) is shown. Particles with poly(I:C) within the backbone were also generated. The formation of the particles was confirmed using scanning electron microscopy (FIG. 4B). It was also confirmed that the particles were phagocytosable using dynamic light scattering analyses (average size=2 μm) (FIG. 4C).

F16BP particles rescues glycolysis even in the presence of glycolytic inhibitor PFK15. In order to test if F16BP can indeed functionally accelerate glycolysis in the presence of low glucose environment, extracellular acidification rate (ECAR), which measures changes in extracellular pH due to lactic acid production (a byproduct of glycolysis) was determined using Seahorse assays. In this case, although, C57BL/6j bone marrow derived dendritic cells (DCs, another type of phagocytic cell) were utilized, it is believed that CAR macrophages will behave similarly. Specifically, DCs were treated with PFK15 (200 nM), or F16BP particles (0.1 mg/mL) or PFK15+F16BP particles. After 4 hours of treatment with formulations, changes in pH using ECAR were recorded in glucose-free media. In a step-wise manner cells were added with oligomycin to measure ATP production and proton leak, Carbonyl cyanide-4 (trifluoromethoxy) phenylhydrazone (FCCP—disrupts mitochondrial membrane potential) to measure maximal respiration, rotenone and antimycinA (shuts down mitochondrial respiration) to measure spare capacity. It was observed that PFK15 brought the ECAR values even lower than no treatment control. ECAR values were significantly higher in F16BP particles with PFK15 group, than PFK15 alone control. These data indicate that F16BP particles can rescue the glycolysis in DCs in the presence of PFK15.

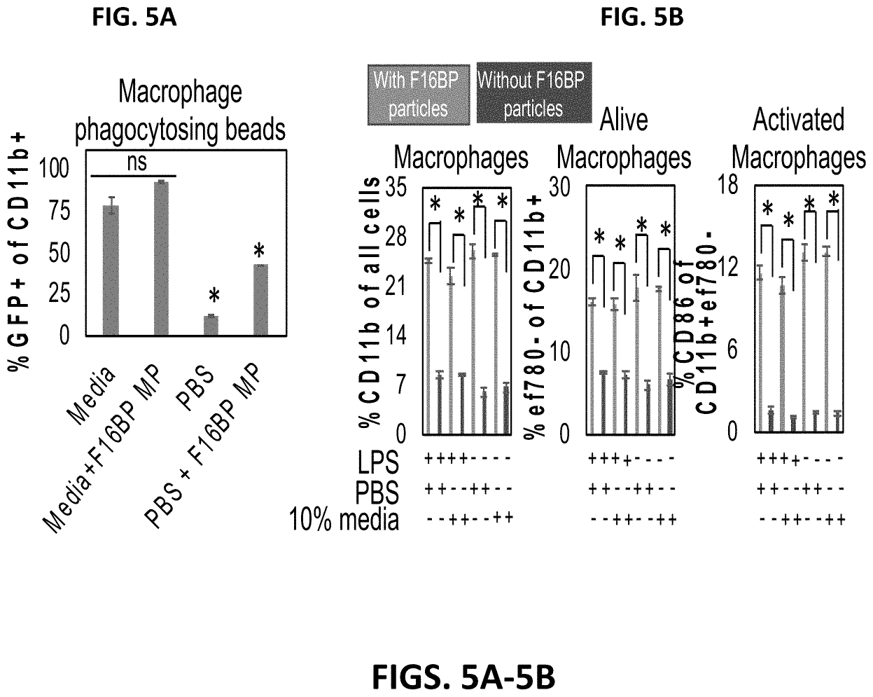

Even in nutrient poor environment, F16BP particles help human macrophages remain alive, activated and perform their phagocytosis function. To test if F16BP particles (without poly(I:C)) can maintain phagocytic function of human macrophages in nutrient free environment, human macrophages were differentiated from monocytes isolated from blood using an 8-day rhGMCSF protocol. These macrophages were then incubated with media or PBS in the presence or absence of the F16BP particles for 16 hours. Fluorescein dye (GFP channel on flow cytometry) containing polystyrene beads were added to this culture for 2 hours, and cells were then washed and stained for CD11b antibodies for flow cytometry analyses. FIG. 5A demonstrates that even in the absence of nutrients (phosphate buffered saline—PBS) F16BP microparticles were able to induce phagocytosis of beads. Also, F16BP particles did not hinder the ability of macrophages (identified as CD11b+) to phagocytose beads in media.

To test if F16BP particles can prevent cell death and maintain function of human macrophages in nutrient-poor environment, macrophages were incubated with F16BP particles in PBS or 10% media, in the presence or absence of LPS (lipopolysaccharide-activating agent) for 16 hours. These macrophages were then stained with live/dead dye, CD11b and CD86 (activation marker) and analyzed using flow cytometry. FIG. 5B demonstrates that the frequency of macrophages was 3-4-fold higher in F16BP group as compared to the condition without F16BP particles. Importantly, the frequency of alive macrophages was 2-3-fold higher, and activated macrophages were 12-15-fold higher in macrophages, in the group with F16BP particles in nutrient-poor environment. These data demonstrate that F16BP particles are able to not only keep macrophages alive, but they also lead to elevated function in nutrient-poor environment.

### Example 2

### F16BP Particle-Loaded CAR-Macs can Target Solid Lymphoma Tumors in Mice

This Example shows that primary CAR-macs infiltrate solid lymphoma tumor in mice and clear these tumors.

CAR expression in macrophage-like cells. To test if the CAR-macs can be generated using non-viral methods, RAW 264.7 murine macrophage-like cells, were transfected using lipofectamine with the plasmids described in scientific rigor section. Expression of GFP was determined using fluorescent microscope and flow cytometry. The GFP⁺ cells were selected under geneticin stress selection. It was determined that approximately 96±0.3% of these cells were positive for Tandem and 97.7±0.6% were positive for Empty plasmids.

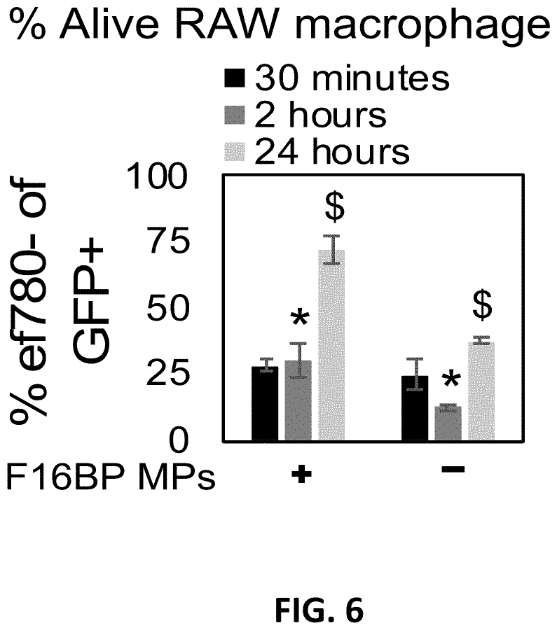

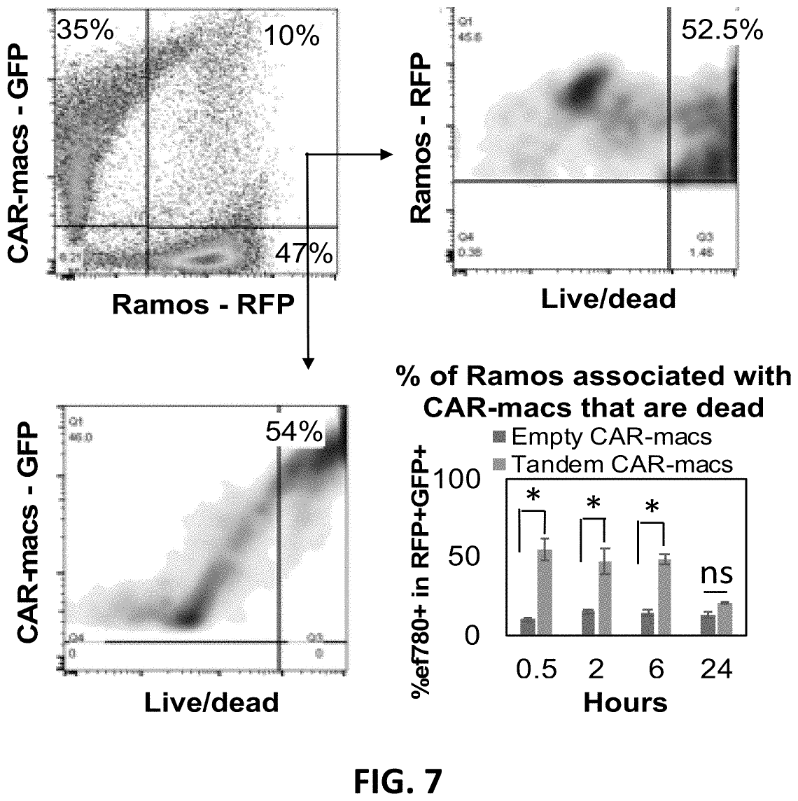

In nutrient-poor environment, CAR-macs incubated with F16BP particles survive in higher numbers. To determine if F16BP particles can prevent RAW macrophage-like cell death, RAW macrophages were incubated with or without F16BP particles in PBS and with or without Ramos cells. These cells were then stained with viability dye, and analyzed using flow cytometry. FIG. 6 demonstrates that the frequency of alive macrophages was 2-3 fold higher at 2 and 24 hour of coculture (*,$), in the F16BP particles group as compared to controls. Interestingly, at 24 hour time point the % alive cell increased, indicating Ramos cells might have been used as fuel-source, and the negative control of without Ramos and without particles Tandem CAR-macs phagocytose higher number of Ramos-RFP cells than Empty CAR-macs. To test if Tandem CAR expression indeed leads to higher phagocytosis of CD19⁺ lymphoma cells, Tandem or Empty CAR expressing RAW macrophages were incubated with Ramos-RFP expressing cells for different periods of time. Percentage death was measure by staining with ef780 cell staining dye. FIG. 7 (graph bottom right) demonstrates that at 0.5, 2 and 6 hours, Tandem expressing RAW CAR-macs were able to induce higher percentage (5-10-fold) of cell death in Ramos cells. However, this significance was lost at 24-hour time point, potentially due to higher proliferation rate of Ramos cells in vitro. These data demonstrate that the Tandem CAR-macs can prevent tumor cell growth.

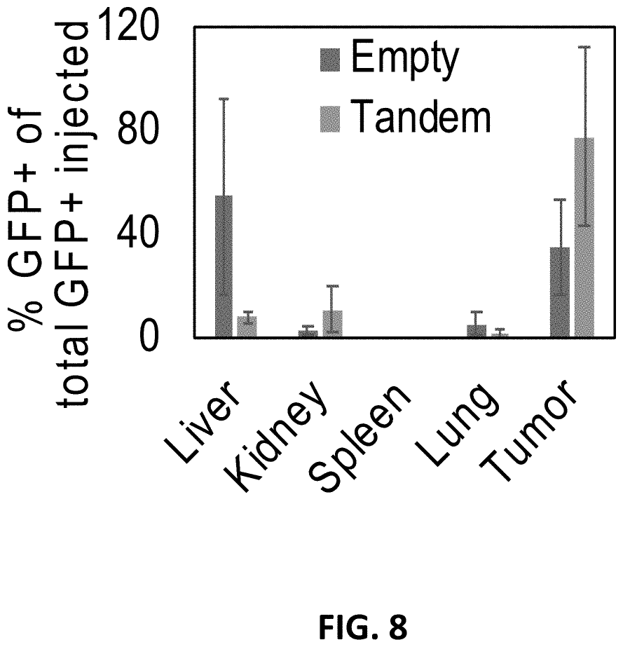

Tandem RAW CAR-macs home to solid lymphoma tumors in NSG mice. To test the if CAR-macs can home to the site of solid tumors, 1×10⁶ Ramos were injected subcutaneously in the back of the nod scid gamma (NSG) mice. Once the tumor was palpable (˜20 days) mice were injected with 0.5×10⁶ Tandem CAR-macs or Empty CAR-macs or saline (control). Mice were sacrificed after 24 hours; major organs were isolated and cells in these organs were analyzed for the presence of GFP using flow cytometry. FIG. 8 shows the biodistribution of the CAR cells in different organs, which indicates that Tandem CAR-macs preferentially home to the solid lymphoma tumors. Higher level of Tandem CAR-macs localized in tumors as compared to Empty CAR-macs potentially due to upregulation of adhesion and spreading signals in Tandem CAR-macs upon CD19-scFv interaction.

### Example 3

### Electroporation Induced CAR Expression in Neutrophils Differentiated from HL-60 Cell Line

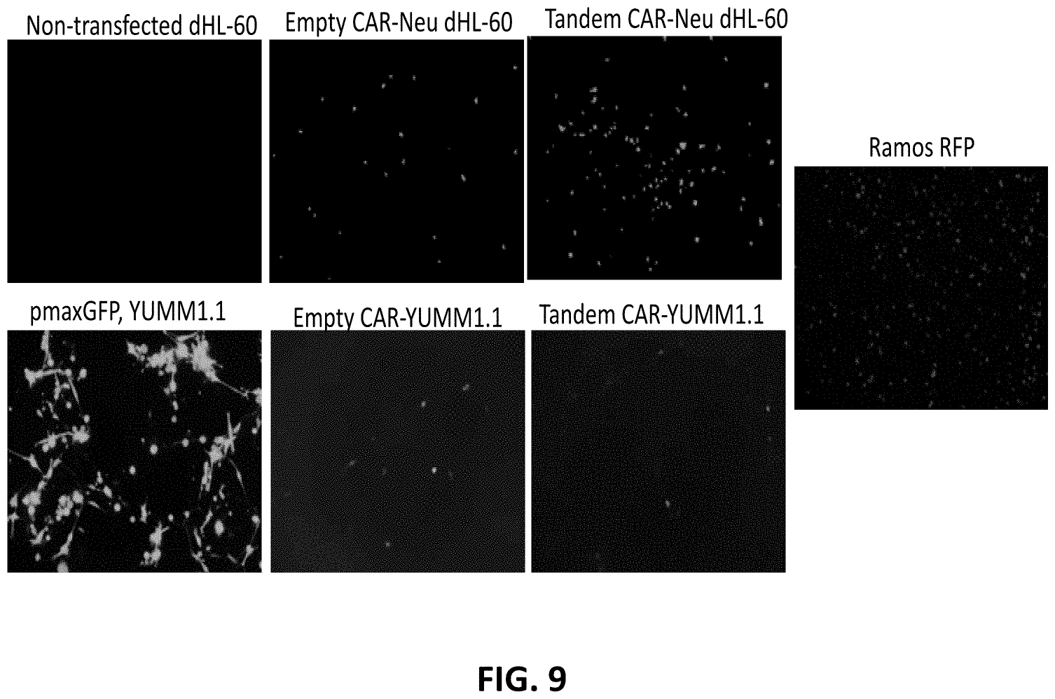

In order to test if the CAR-Neu can be generated, first HL-60, a leukemia cell line, was differentiated into neutrophil-like cells using 1.3% of DMSO treatment over 5 days. Next, these cells were transfected with the plasmids described herein using AMAXA® electroporator system. The expression of GFP was determined using fluorescent microscope and flow cytometry (FIG. 9). A plasmid capable of inducing GFP expression alone was used as control. Moreover, an adherent melanoma cell line YUMM1.1 was utilized as a control as well. It was determined that approximately 8±0.3% of the differentiated HL-60 (dHL-60) cells could be transfected with the Tandem and 7.7±0.6% with empty plasmids. Lastly, a stable RFP expressing Ramos B-cell lymphoma cell line was also used for this project. These data indicate that differentiated HL-60 (dHL-60) cells can be transfected with CAR plasmids and thus can be utilized to study the interaction with CD19 expressing B-cell lymphoma, such as Ramos cell line.

### Example 4

### CAR-Neu Cells Associate with Ramos-RFP Cells In Vitro, and Tandem CAR-Neu Cells Phagocytose/Trogocytose Higher Number of Ramos-RFP Cells than Empty CAR-Neu Cells

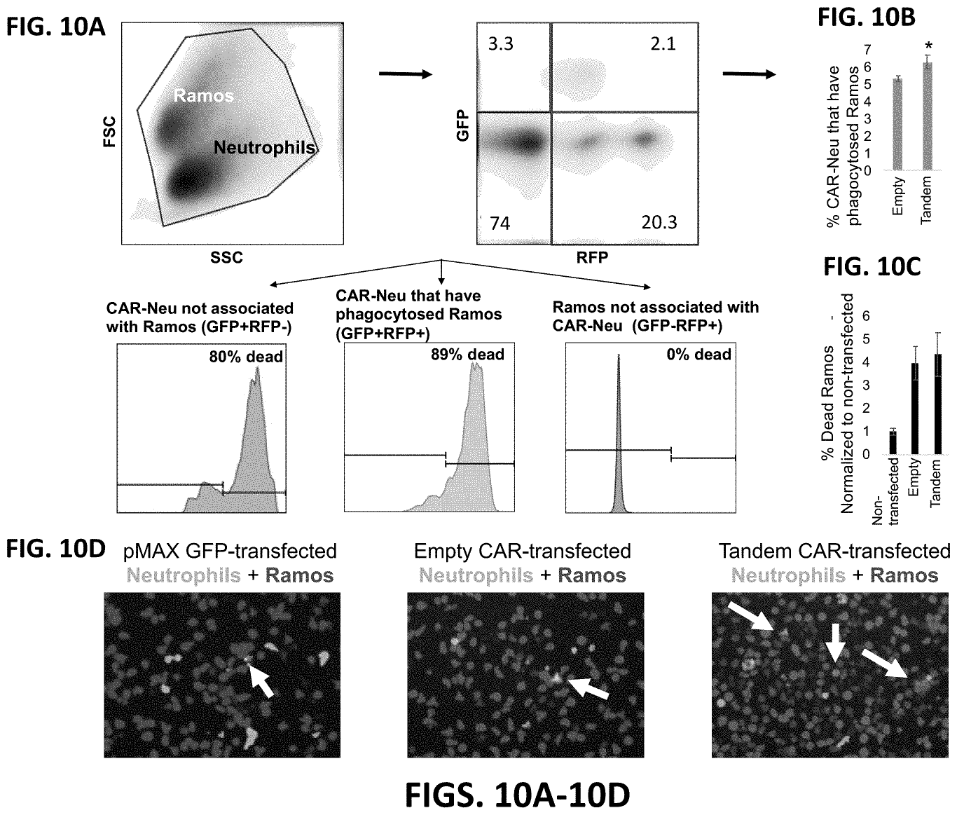

In order to test if dHL-60 CAR-Neu cells can phagocytose Ramos-RFP cells, the two cells were incubated together. Specifically, dHL-60 CAR-Neu cells were generated by electroporation with Tandem or Empty plasmids and immediately added to the Ramos-RFP cells in a 24 well plate at 1 to 2 ratio (HL-60 to Ramos). The cells were co-cultured for 6 hours and then stained with live/dead 780 dye for evaluating number of dead cells and fixed using 4% PFA. These cells were then analyzed using flow cytometry (FIG. 10A). Notably, there were significantly higher levels of Ramos cells that associated with Tandem CAR-Neu, as compared to empty CAR-Neu (FIG. 10B), and high levels (approximately 89%) of neutrophils associated with Ramos cells were dead. Both Tandem and Empty CAR-Neu cells led to 4 fold higher killing of Ramos cells as compared to non-transfected neutrophils (FIG. 10C). FIG. 10D shows fluorescent microscope images of CAR-Neu cells (GFP) interacting with Ramos lymphoma (RFP) cells. Overall, these data indicate that CAR-Neu cells can not only strongly associate with Ramos cells, but also lead to higher levels of killing of lymphoma cells.

### Example 5

### Tandem CAR-Neu Cells Secrete NETs when Cultured with Ramos Cells

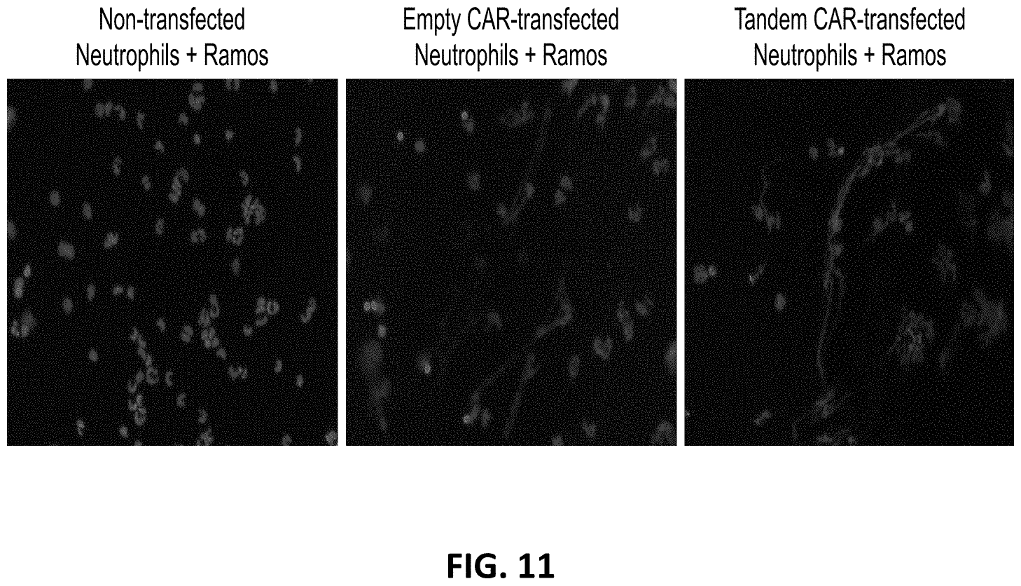

In order to determine if CAR-Neu cells which potentially express the PI3K recruiting intracellular domain, responsible for neutrophil activation, can induce NETosis in these cells, Tandem CAR-Neu or Empty CAR-Neu cells were incubated with Ramos cells (same as Examples above). After 6 hours of incubation cells were removed, and the plates were washed using 0.1% tween20 in phosphate buffered saline solution to remove any unbound cells or cell debris. Next, the plate surface was incubated with DAPI to stain for DNA in the NETs secreted by neutrophil-like cells. It was observed that NETs were generated by neutrophils that were transfected with Tandem CAR plasmid and empty CAR plasmid (FIG. 11). These data indicate that CAR-Neu cells area capable of generating NETs when cultured with Ramos cells.

In view of the many possible embodiments to which the principles of the present disclosure may be applied, it should be recognized that the illustrated embodiments are only preferred examples of the invention and should not be taken as limiting the scope of the invention. Rather, the scope of the invention is defined by the following claims.

1. A composition comprising:
a chimeric antigen receptor (CAR) comprising an extracellular domain comprising a single chain variable fragment (scFv) that binds CD19, CD22, mesothelin, CA-125, or HER2;
a cytoplasmic domain comprising a costimulatory domain and a signaling domain; and
a glycolysis accelerating metabolite.

2. The composition of claim 1, wherein the extracellular domain comprising the scFv and/or the cytoplasmic domain comprising the costimulatory domain and the signaling domain are expressed in an immune cell by delivery of mRNA or plasmid DNA encoding the extracellular domain and/or the cytoplasmic domain.

3. The composition of claim 1, wherein the glycolysis accelerating metabolite is in particle form.

4. The composition of claim 1, wherein the glycolysis accelerating metabolite is in particle form that encapsulates and releases one or more adjuvants in a controlled manner.

5. The composition of claim 1, wherein the costimulatory domain comprises an intracellular p85-mediated PI3K recruiting domain or a CD3zeta domain.

6. The composition of claim 1, wherein the signaling domain comprises an FcRgamma, a 4-1BB, a CD28, and/or an ICOS signaling domain.

7. The composition of claim 1, wherein the glycolysis accelerating metabolite comprises F6P, G6P, PVP, F16BP, or succinate.

8. The composition of claim 1, wherein the composition is expressed in antigen presenting cells, macrophages, dendritic cells, or neutrophils

9. (canceled)

10. An isolated nucleic acid molecule encoding the CAR in the composition of claim 1.

11. A vector comprising the nucleic acid molecule of claim 10.

12. A cell comprising the nucleic acid molecule of claim 10.

13. A cell comprising the vector of claim 11.

14. The cell of claim 13, wherein the cell is a human antigen presenting cell, macrophage, human T cell, NK cell, or human neutrophil.

15. (canceled)

16. (canceled)

17. An engineered cell comprising the composition of claim 1.

18. The engineered cell of claim 17, wherein the engineered cell is an immune cell or an immune effector cell.

19. (canceled)

20. The engineered cell of claim 18, wherein the immune effector cell is a T cell, an NK cell, or a macrophage.

21. (canceled)

22. The engineered cell of claim 17 for use in the treatment of a solid tumor, lymphoma and/or leukemia tumors.

23. (canceled)

24. A pharmaceutical composition comprising:
a genetically-modified human macrophage comprising a chimeric antigen receptor (CAR) comprising an extracellular domain comprising a single chain variable fragment (scFv) that binds CD19, CD22, mesothelin, CA-125, or HER2, and a cytoplasmic domain comprising a costimulatory domain and a signaling domain; and
a glycolysis accelerating metabolite.

25. A method for treating a subject suffering from a solid or diffused tumor, comprising introducing into the subject a therapeutically effective amount of the pharmaceutical composition of claim 24.

26. The method of claim 25, wherein the solid or diffused tumor is a lymphoma and/or leukemia and/or melanoma and/or ovarian tumor.
